SEQUENCE LISTING (101 sequences)

SEQ ID NO: 1 - protein, 5 aa, synthetic construct
GGGGS

SEQ ID NO: 2 - protein, 4 aa, synthetic construct
GGSG

SEQ ID NO: 3 - protein, 4 aa, synthetic construct
SGGG

SEQ ID NO: 4 - protein, 15 aa, synthetic construct
GGGGSGGGGSGGGGS

SEQ ID NO: 5 - protein, 14 aa, synthetic construct
GGGGDGGGGDGGGG

SEQ ID NO: 6 - protein, 14 aa, synthetic construct
GGGGEGGGGEGGGG

SEQ ID NO: 7 - skipped

SEQ ID NO: 8 - protein, 40 aa, synthetic construct
LCQPCRSPRWSLWSTWAPCSVTCSEGSQLRYRRCVGWNGQ

SEQ ID NO: 9 - protein, 10 aa, synthetic construct
GRIFEVNMMA

SEQ ID NO: 10 - protein, 13 aa, synthetic construct
  VARIANT 6: note = MISC_FEATURE - Xaa is Ala, Cys, Phe, Gly, His, Ile, Leu, Met, Asn, Pro, Gln, Ser, Thr, Val, Trp, or Tyr
RVGTTXYADSVKG

SEQ ID NO: 11 - protein, 11 aa, synthetic construct
  VARIANT 4: note = MISC_FEATURE - Xaa is Asn, Cys, Gln, His, Met, Ser, Thr, Trp, or Tyr
LQYXRYGGAEY

SEQ ID NO: 12 - protein, 13 aa, synthetic construct
RVGTTVYADSVKG

SEQ ID NO: 13 - protein, 11 aa, synthetic construct
LQYDRYGGAEY

SEQ ID NO: 14 - protein, 11 aa, synthetic construct
LQYSRYGGAEY

SEQ ID NO: 15 - protein, 13 aa, synthetic construct
RVGTTTYADSVKG

SEQ ID NO: 16 - protein, 10 aa, synthetic construct
GRISSIIHMA

SEQ ID NO: 17 - protein, 11 aa, synthetic construct
LQYEKHGGADY

SEQ ID NO: 18 - protein, 10 aa, synthetic construct
GYIFTNYPIH

SEQ ID NO: 19 - protein, 17 aa, synthetic construct
FIDPGGGYDEPDERFRD

SEQ ID NO: 20 - protein, 9 aa, synthetic construct
RGGGYYLDY

SEQ ID NO: 21 - protein, 11 aa, synthetic construct
RASQDISFFLN

SEQ ID NO: 22 - protein, 7 aa, synthetic construct
YTSRYHS

SEQ ID NO: 23 - protein, 9 aa, synthetic construct
QHGNTLPWT

SEQ ID NO: 24 - protein, 10 aa, synthetic construct
GFSLTTYGVH

SEQ ID NO: 25 - protein, 16 aa, synthetic construct
VIWSGGDTDYNASFIS

SEQ ID NO: 26 - protein, 14 aa, synthetic construct
NKDYYTNYDFTMDY

SEQ ID NO: 27 - protein, 17 aa, synthetic construct
KSSQSVLYSSNQKNFLA

SEQ ID NO: 28 - protein, 7 aa, synthetic construct
WASTRES

SEQ ID NO: 29 - protein, 8 aa, synthetic construct
HQYLSSYT

SEQ ID NO: 30 - protein, 10 aa, synthetic construct
GYTFIDYWIE

SEQ ID NO: 31 - protein, 17 aa, synthetic construct
EIFPGSGTINHNEKFKD

SEQ ID NO: 32 - protein, 5 aa, synthetic construct
EGLDY

SEQ ID NO: 33 - protein, 10 aa, synthetic construct
SASSSVSYIY

SEQ ID NO: 34 - protein, 7 aa, synthetic construct
DTSTLAS

SEQ ID NO: 35 - protein, 9 aa, synthetic construct
QQWSRNPFT

SEQ ID NO: 36 - protein, 10 aa, synthetic construct
GFSLTSYGVH

SEQ ID NO: 37 - protein, 16 aa, synthetic construct
VIWSGGSTDYNAAFIS

SEQ ID NO: 38 - protein, 14 aa, synthetic construct
NKDFYSNYDYTMDY

SEQ ID NO: 39 - protein, 10 aa, synthetic construct
  VARIANT 4: note = misc_feature - Xaa can be any naturally occurring amino acid
GYTXTAYGIN

SEQ ID NO: 40 - protein, 17 aa, synthetic construct
YIYIGNGYTDYNEKFKG

SEQ ID NO: 41 - protein, 11 aa, synthetic construct
SGWDEDYAMDF

SEQ ID NO: 42 - protein, 11 aa, synthetic construct
RASENIYSYLA

SEQ ID NO: 43 - protein, 7 aa, synthetic construct
HAKTLAE

SEQ ID NO: 44 - protein, 9 aa, synthetic construct
QHHYGPPPT

SEQ ID NO: 45 - protein, 258 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSLEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 46 - protein, 257 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 47 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 48 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSS

SEQ ID NO: 49 - protein, 257 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 50 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 51 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLLESGGGLVQPGGSLRLSCAASGRISSIIHMAWFRQAPGKERELVSEISRVGTTVYADSVKGRFTISRDNSKNTLYLQMNSLKPEDTAVYYCNALQYEKHGGADYWGQGTLVTVSS

SEQ ID NO: 52 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGDGGGGDGGGGEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 53 - protein, 347 aa, synthetic construct
LEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSSRKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 351 aa, synthetic construct
LEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 258 aa, synthetic construct
LEVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 56 - protein, 214 aa, synthetic construct
DIQMTQSPSSLSASVGDRVTITCRASQDISFFLNWYQQKPGKAPKLLIYYTSRYHSGVPSRFSGSGSGTDFTLTISSLQPEDFATYYCQHGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 448 aa, synthetic construct
QVQLVQSGAEVKKPGASVKVSCKASGYIFTNYPIHWVRQAPGQGLEWMGFIDPGGGYDEPDERFRDRVTMTRDTSTSTVYMELSSLRSEDTAVYYCARRGGGYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 119 aa, synthetic construct
QVQVVESGGGLRQTGGSLRLSCTASGRIFEVNMMAWYRQAPGKQRELVAEISRVGTTVYADSVKGRFTISRDSAKNTVTLQMNSLKSEDTAVYYCNALQYDRYGGAEYWGQGTQVTVSS

SEQ ID NO: 59 - protein, 119 aa, synthetic construct
QVQLAESGGGLVQAGDSLKLSCTASGRIFEVNMMAWYRQAPGKDRELVAEISRVGTTTYADSVKGRFTISRDSAKNTVTLQMNSLKSEDTAVYYCNALQYSRYGGAEYWGQGTQVTVSG

SEQ ID NO: 60 - protein, 119 aa, synthetic construct
QVQLVESGGGLVQAGGSLRLSCAASGRISSIIHMAWYRQAPGKQRELVAEISRVGTTVYADSVKGRFTISRDDAKNTVTLQMNSLKPEDTAVYYCNALQYEKHGGADYWGQGTQVTVSG

SEQ ID NO: 61 - protein, 119 aa, synthetic construct
QVQLVESGGGLRQTGESLRLSCTASGRIFEVNMMAWYRQAPGKQRELVAEISRVGTTTYADSVKGRFTISRDSAKNTVTLQMNSLKSEDTAVYYCNALQYDRYGGAEYWGQGTQVTVSG

SEQ ID NO: 62 - protein, 256 aa, synthetic construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGEGGGGEGGGGEVQLVESGGGLVQPGGSLRLSCAASGRISSIIHMAWVRQAPGKQRELVSEISRVGTTVYADSVKGRFTISRDNSKNTLYLQMNSLRAEDTAVYYCNALQYEKHGGADYWGQGTLVTVSS

SEQ ID NO: 63 - protein, 5 aa, synthetic construct
GGGGD

SEQ ID NO: 64 - protein, 5 aa, synthetic construct
GGGGE

SEQ ID NO: 65 - DNA, 40 bp, synthetic construct
  source 1..40: mol_type = other DNA
gtcaccgtgtcgagccatcatcaccatcatcactgatgag

SEQ ID NO: 66 - DNA, 45 bp, synthetic construct
  source 1..45: mol_type = other DNA
aattctcatcatttgtcatcatcatccttatagtcgctcgacacg

SEQ ID NO: 67 - DNA, 36 bp, synthetic construct
  source 1..36: mol_type = other DNA
gtccactccctcgaggtgcagctggtggagtctggg

SEQ ID NO: 68 - DNA, 33 bp, synthetic construct
  source 1..33: mol_type = other DNA
gctcgacacggtgacctgggtcccctggcccca

SEQ ID NO: 69 - DNA, 27 bp, synthetic construct
  source 1..27: mol_type = other DNA
cataatagctgacagactaacagactg

SEQ ID NO: 70 - DNA, 27 bp, synthetic construct
  source 1..27: mol_type = other DNA
cgaaacaagcgctcatgagcccgaagt

SEQ ID NO: 71 - protein, 122 aa, synthetic construct
QVQLVESGGGLVQAGGSLRLSCAASGRTFSSYAMGWFRQAPGKEREFVAAIGWNGEGIYYADSVKGRFTISRDNAKNTGYLQMNSLKPEDTAVYYCAADSEGVVPGFPIAYWGQGTQVTVSG

SEQ ID NO: 72 - protein, 129 aa, synthetic construct
QVQLVESGGGLVQPGGSLRLSCAASGFPLNSYAIGWFRQAPGKEREGVSCISVSDDSTYYTDSVKGRFTISRDNAKNTVYLQMNSLKPEDTAVYYCAVDSAPLYGDYVCKPLENEYDFWGQGTQVTVSG

SEQ ID NO: 73 - protein, 118 aa, synthetic construct
  VARIANT 19: note = misc_feature - Xaa can be any naturally occurring amino acid
QVQLVESGGGLVQAGGSLXLSCAASGSDRRINGMGWYRHPPGKQRELVAAITSGGSTNYADSVKGRFTISTNNANNMMYLQMNSLKPEDTAVYYCAIDEFGTGWLDYCGQGTQVTVSG

SEQ ID NO: 74 - protein, 122 aa, synthetic construct
QVQLVESGGGLVQPGGSLRLSCAASGRPFSSYAMGWFRQAPGKEREIVAGLSWSGGNVYYADSVKGRFTISRDNAKNTGDLQMNSLKPEDTAVYYCAIGPKLTTGPTAYRYWGQGTQVTVSS

SEQ ID NO: 75 - protein, 129 aa, synthetic construct
QVQLVESGGGLVQPGGSLRLSCATSGGTFSSYAMGWFRQAPGKEREFVAAITWNGSNRYYADSVKGRFTISRDNAKSTVYLQMNSLKPEDTAVYYCAAEHSTRYSGFYYYTRGETYHYWGQGTQVTVSG

SEQ ID NO: 76 - protein, 129 aa, synthetic construct
QVQLVESGGGLVQAGGSLRLSCAASGRTFSTLGMGWFRQAPGKERQFVAAINWSGSSTYYANSVKGRFTISRDNAQSTMYLQMNSLKPEDTAVYYCAADLDSRYSAYYYYSDESQYDYWGQGTLVTVSG

SEQ ID NO: 77 - protein, 129 aa, synthetic construct
QVQVVESGGGLVQPGGSLSLSCAASGRTFSSYAMGWFRQAPGKEREFVAAITWDGANIYYADSVKGRFTLSRDNAENTVWLQLNSLKPEDTAVYYCAAAESGRYSGRDYYSAPGVYLYWGQGTLVTVSG

SEQ ID NO: 78 - protein, 129 aa, synthetic construct
QVQLVESGGGLVQAGGSLRLSCAASGSIFDINAMGWYRQAPGKQRELVADITSSGSTNYADSVKGRFTISRDNAKNTVYLQMNSLKPEDTAVYTCAAESIRESQNRHQLGYMGPLYDYWGQGTQVTVSG

SEQ ID NO: 79 - protein, 129 aa, synthetic construct
QVQLIESGGGLVQAGDSLRLSCAASEGTFSRFAMGWFRQAPGKEREFVAAINWSGGITYYADSVKGRFTISRDNAKNTVYLQMNSLKPEDTADYYCAAETTTRYSGYYYYEDNKSYDYWGQGTLVTVSG

SEQ ID NO: 80 - protein, 120 aa, synthetic construct
QVLLEESGGGLERTGGSLRLSCAASGSIFSVNSMTWYRQAPGKRREFLGTITEEGRTNYADSVKGRFTISRDNAKNTMYLQMNSLKPEDTAVYYCYANLISSEDRTFGVWGQGTQVTVSS

SEQ ID NO: 81 - protein, 122 aa, synthetic construct
QVHLVESGGGLVQAGGSLRLSCTASGGTVGDYAVGWFRQAPGKERELIGVVSRLGARTGYADSVLGRFTISRDDVKNTVFLQMDSVKPEDTAVYYCAARRDYSFEVVPYDYWGQGTQVTVSG

SEQ ID NO: 82 - protein, 116 aa, synthetic construct
QVQMVESGGGLVQAGGSLRLSCAASGLTNRIRIMGWYRQAPGKLRELVATITNDGSTHYADSVKGRFTISTDNAKNTVFLQMNSLKPEDTAVYICNVGENWGPAYWGQGTQVTVSG

SEQ ID NO: 83 - protein, 128 aa, synthetic construct
QVRLTESGGGLVQYGTNLTLTCVASGLISTRNKMGWFRRRSGGQREFVASSTVLSDDVIQDDIAETVKGRFAVARNDYKNILYLQMTAVKPEDTGFYWCASGTSLFGASRREDDFNAWGVGTQVTVSA

SEQ ID NO: 84 - protein, 119 aa, synthetic construct
EVQLVESGGGLVQPGGSLRLSCAASGFTFGSADMSWVRQAPGKGPEWVSAINSNGGSTYYAASVKGRFTISRDNAKNTLYLQMNSLKPEDTAVYYCAQGNWYTEEYHYWGQGTLVTVSG

SEQ ID NO: 85 - protein, 129 aa, synthetic construct
QVRLVESGGGLVQAGDSLRLSCAASGRTLSSYAMGWFRQAPGKEREFVAATTWRDTSTYYADSVKGRFTISRDNAKNTVYLQMNSLKPEDTAAYYCAAEEPSKYSGRDYYMMGDSYDYWGQGTQVTVSS

SEQ ID NO: 86 - protein, 119 aa, synthetic construct
EVQLVESGGGLVQPGGSLRLSCAASGFTFGSADMSWVRQAPGKGPEWVSAINSNGGSTYYAASVKGRFTISRDNAKNTLYLQMNSLKPEDTAVYYCAQGNWYTEEYHYWGQGTQVTVSG

SEQ ID NO: 87 - protein, 129 aa, synthetic construct
QVQLVESGGGLVQAGGSLRLSCAASGRTFSNYAMAWFRQAPGKEREFVASISGSGDSRYYADSVKGRFTISRDNAKNTVYLQTNSPKPEDTAVYYCAAVLPTRYSGFYYYSDGTQYHYWGQGTQVTVSS

SEQ ID NO: 88 - protein, 118 aa, synthetic construct
QVNLVESGGGSVQAGGSLRLSCAASENINVINDMGWYRQAPGKQRELVAVITGHDNINYADSATGRFTISTYTWNTENLQMNMLKPEDTAVYYCNADITYANGRFNDWGQGTQVTVSS

SEQ ID NO: 89 - protein, 129 aa, synthetic construct
QVHLVESGGGLVQAGGSLRLSCAASGRTFSSYAMGWFRQPPGKEREFVAAITWSGSSIYYADSVKGRFTISRDNAKNTVYLQMNSLKPEDTAVYYCAAEETSKYSGSYYYMMGDSYDYWGQGTQVTVSG

SEQ ID NO: 90 - protein, 130 aa, synthetic construct
QVQLVESGGGLVQAGGSLRLSCAASGRTFSSYAMGWFRQAPGKEREFVAAVPWTYGSKYYADSVKGRFTISRDDAKNTVYLQMNNLKPEDTAVYYCAADSSAGYYSGFDYYSAATPYDLWGQGTQVTVSG

SEQ ID NO: 91 - protein, 125 aa, synthetic construct
QVQLVESGGGLVQPGGSLRLSCAASGSDYYAIGWFRQAPGKEREGVSCMSRTDGSTYYADSVKDRFTISRDYAKNTVYLQMNSLKPEDTAVYYCGLDRSYPTGGISCLFGDFGSWGQGTQVTVSG

SEQ ID NO: 92 - protein, 124 aa, synthetic construct
QVQLVESGGGLVQAGGSLRLSCAASGRTFSSYNMGWFRQRHGNEREFVATISWSGRSTYYADSVKGRFAISRDNANTTVYLQMNSLKPEDSAVYYCAASTRGWYGTQEDDYNFWGQGTQVTVSG

SEQ ID NO: 93 - protein, 129 aa, synthetic construct
QVQLVESGGGLVQAGGSLRLSCAASGGTFSSYSMGWFRQAPGKEREFVAAITWNGVSTYYADSVKGRFTISRDNAKNTVYLQMNSLKPTDTAVYYCAAEITTRYSGFYYYEDNKSYDYWGQGTQVTVSS

SEQ ID NO: 94 - protein, 127 aa, synthetic construct
EVQLVESGGGLVQPGGSLRLSCAASGFTLDYYAIGWFRQAPGKEREGVSCISRTDGSTYYADSVKGRFTISRDNAKNTVYLQMNSLKPEDTAVYYCAVDDSYPTGGISCLFGHFGSWGQGTQVTVSS

SEQ ID NO: 95 - protein, 129 aa, synthetic construct
QVQLVESGGGLVQAGDSLRLSCAASGFTFSSYAMGWFRQAPGKEREFVAAITWSGVSTYYADSVKGRFTISRDNAKNRVYLQMNSLKPEDTAVYSCAADGSGRYSGMEYYNRDWVYDYWGQGTQVTVSS

SEQ ID NO: 96 - protein, 121 aa, synthetic construct
QVHMVESGGGLVQAGGSLRFSCAASGNIFTISTLDWYRQAPGEQRELVATLTPDGITDYAGSVKGRFTISRDNAKNTVYLQMNSLKPEDTAVYYCNAWRYSDDYRGRVDYWGQGTQVTVSG

SEQ ID NO: 97 - protein, 126 aa, synthetic construct
QVQLIESGGGLVQEGASLRLSCAGSGPMFSRLAVGWFRQAPGKEREFVAVINWSGSADFYTNSVKGRFTISRDNAKNTVYLEMNTLKPEDSAVYYCAADQNPLTLRTGVRDVGRQWGQGTEVTVSS

SEQ ID NO: 98 - protein, 129 aa, synthetic construct
QVQLVESGGGLVQAGGSLRLSCAASGRTFSSYAMGWFRQAPGKEREFVAAITWRGASTYYADPVKGRFTISRDNAKNTVYLQMSSLKPEDTAVYYCAAEEPSYYSGSYYYMMGDSYNYWGQGTQVTVSG

SEQ ID NO: 99 - protein, 125 aa, synthetic construct
QVQLVESGGGLVQAGGSLRLSCTASGRTFSNYAMGWFRQAPGKEREFLAAISRSGESTNYATFVKGRFTIARDNAKNTVSLQMNSLKPEDTAVYFCAAKVAVLVSTTYNSQYDYWGQGTQVTVSS

SEQ ID NO: 100 - protein, 5 aa, synthetic construct
GGGGA

SEQ ID NO: 101 - protein, 15 aa, synthetic construct
GGGGAGGGGAGGGGS

(19) United States
(12) Patent Application Publication
(10) Pub. No.: US 2026/0070963 A1
(43) Pub. Date: Mar. 12, 2026

(54) MONOVALENT ANTI-PROPERDIN ANTIBODIES AND ANTIBODY FRAGMENTS

(71) Applicant: Alexion Pharmaceuticals, Inc., Boston, MA (US)

(72) Inventors: Douglas L. SHERIDAN, Branford, CT (US); Paul P. TAMBURINI, Kensington, CT (US); Taneisha Ann-Tanara MACK, East Hampton, CT (US); Walter C. VOEGTLI, Killingworth, CT (US)

(21) Appl. No.: 19/387,895

(22) Filed: Nov. 13, 2025

Related U.S. Application Data
(63) Continuation of application No. 17/522,028, filed on Nov. 9, 2021, now Pat. No. 12,497,445.
Application No. 17/522,028 is a continuation of application No. 16/479,335, now Pat. No. 11,198,725.
(60) Provisional application No. 62/452,187, filed on Jan. 30, 2017.

(51) Int. Cl. C07K 16/18

(52) U.S. Cl. CPC C07K 16/18; C07K 2317/22; C07K 2317/24; C07K 2317/565; C07K 2317/569; C07K 2317/92

(57) ABSTRACT

Described herein are isolated monovalent antibodies or antibody fragments thereof that bind human properdin. Such antibodies are useful in methods of treatment for diseases mediated by alternative complement pathway dysregulation.

### SEQUENCE LISTING

The instant application contains a Sequence Listing which has been submitted electronically in XML format and is hereby incorporated by reference in its entirety. Said XML copy, created on Nov. 12, 2025, is named 51196-003004_Sequence_Listing_11_12_25.xml and is 97,274 bytes in size.

### BACKGROUND

The complement system plays a central role in the clearance of immune complexes and in immune responses to infectious agents, foreign antigens, virus-infected cells and tumor cells. Complement activation occurs primarily by three pathways: the classical pathway, the lectin pathway and the alternative pathway. Uncontrolled activation or insufficient regulation of the alternative complement pathway can lead to systemic inflammation, cellular injury and tissue damage. The alternative complement pathway has been implicated in the pathogenesis of a growing number of diverse diseases. Properdin positively regulates alternative complement pathway activation by binding and stabilizing the C3 and C5 convertase complexes (C3bBb and C3bnBb). Inhibition or modulation of properdin activity is an important therapeutic strategy to mitigate symptoms and slow progression of diseases associated with alternative complement pathway dysregulation. There continues to be an unmet need for effectively regulating properdin activity.

### SUMMARY

Described herein are isolated monovalent antibodies and antibody fragments thereof that specifically or substantially specifically bind properdin and selectively block alternative complement pathway activation. By inhibiting the functional activity of properdin, the monovalent antibody described herein inhibits alternative complement pathway-induced assembly of the membrane attack complex. In addition, selective binding of a single properdin molecule with a monovalent antibody can reduce undesirable immune complexes, resulting from aggregation. Thus, the selective targeting of a single properdin monomer or multimer can, in turn, improve clinical benefits for patients with disease mediated by alternative complement pathway dysregulation.

In one embodiment, the disclosure is directed to an isolated monovalent antibody or antibody fragment thereof, where the antibody or antibody fragment thereof binds human properdin. In a particular embodiment, the antibody or fragment is a camelid antibody. In a particular embodiment, the antibody or fragment is a single-domain antibody. In a particular embodiment, the antibody or fragment binds to TSR0 and/or TSR1 of human properdin. In a particular embodiment, the antibody or fragment binds an epitope within the amino acid sequence LCQPCRSPRWSLWSTWAPCSVTCSEGSQLRYRRCVGWNGQ (SEQ ID NO: 8). In a particular embodiment, the antibody or fragment binds to mouse properdin with an affinity of less than 50 nM. In a particular embodiment, the antibody or fragment comprises at least one or all three CDRs selected from: a) a CDR-H1 including the amino acid sequence GRIFEVNMMA (SEQ ID NO: 9); b) a CDR-H2 including the amino acid sequence RVGTTX₁YADSVKG (SEQ ID NO: 10), where X₁ is a polar or a nonpolar amino acid; and c) a CDR-H3 including the amino acid sequence LQYX₂RYGGAEY (SEQ ID NO: 11), where X₂ is a polar amino acid. In a particular embodiment, CDR-H2 includes the amino acid sequence RVGTTVYADSVKG (SEQ ID NO: 12). In a particular embodiment, CDR-H3 includes the amino acid sequence LQYDRYGGAEY (SEQ ID NO: 13). In a particular embodiment, CDR-H2 includes the amino acid sequence RVGTTTYADSVKG (SEQ ID NO: 15). In a particular embodiment, CDR-H3 has the amino acid sequence LQYSRYGGAEY (SEQ ID NO: 14). In a particular embodiment, CDR-H3 has the amino acid sequence LQYDRYGGAEY (SEQ ID NO: 13). In a particular embodiment, CDR-H3 has the amino acid sequence LQYSRYGGAEY (SEQ ID NO: 14). In a particular embodiment, the antibody or fragment includes 3 CDRs with the following sequences: a) a CDR-H1 having the amino acid sequence GRISSIIHMA (SEQ ID NO: 16); b) a CDR-H2 having the amino acid sequence RVGTTVYADSVKG (SEQ ID NO: 12); and c) a CDR-H3 having the amino acid sequence LQYEKHGGADY (SEQ ID NO: 17). In a particular embodiment, the antibody includes 6 CDRs with the following sequences: a) a CDR-H1 having the amino acid sequence GYIFTNYPIH (SEQ ID NO: 18); b) a CDR-H2 having the amino acid sequence FIDPGGGYDEPDERFRD (SEQ ID NO: 19); c) a CDR-H3 having the amino acid sequence RGGGYYLDY (SEQ ID NO: 20); d) a CDR-L1 having the amino acid sequence RASQDISFFLN (SEQ ID NO: 21); e) a CDR-L2 having the amino acid sequence YTSRYHS (SEQ ID NO: 22); and f) a CDR-L3 having the amino acid sequence QHGNTLPWT (SEQ ID NO: 23). In a particular embodiment, the antibody includes 6 CDRs with the following sequences: a) a CDR-H1 having the amino acid sequence GFSLTTYGVH (SEQ ID NO: 24); b) a CDR-H2 having the amino acid sequence VIWSGGDTDYNASFIS (SEQ ID NO: 25); c) a CDR-H3 having the amino acid sequence NKDYYTNYDFTMDY (SEQ ID NO: 26); d) a CDR-L1 having the amino acid sequence KSSQSVLYSSNQKNFLA (SEQ ID NO: 27); e) a CDR-L2 having the amino acid sequence WASTRES (SEQ ID NO: 28); and f) a CDR-L3 having the amino acid sequence HQYLSSYT (SEQ ID NO: 29). In a particular embodiment, the antibody includes 6 CDRs with the following sequences: a) a CDR-H1 having the amino acid sequence GYTFIDYWIE (SEQ ID NO: 30); b) a CDR-H2 having the amino acid sequence EIFPGSGTINHNEKFKD (SEQ ID NO: 31); c) a CDR-H3 having the amino acid sequence EGLDY (SEQ ID NO: 32); d) a CDR-L1 having the amino acid sequence SASSSVSYIY (SEQ ID NO: 33); e) a CDR-L2 having the amino acid sequence DTSTLAS (SEQ ID NO: 34); and f) a CDR-L3 having the amino acid sequence QQWSRNPFT (SEQ ID NO: 35). In a particular embodiment, the antibody includes 6 CDRs with the following sequences: a) a CDR-H1 having the amino acid sequence GFSLTSYGVH (SEQ ID NO: 36); b) a CDR-H2 having the amino acid sequence VIWSGGSTDYNAAFIS (SEQ ID NO: 37); c) a CDR-H3 having the amino acid sequence NKDFYSNYDYTMDY (SEQ ID NO: 38); d) a CDR-L1 having the amino acid sequence KSSQSVLYSSNQKNFLA (SEQ ID NO: 27); e) a CDR-L2 having the amino acid sequence WASTRES (SEQ ID NO: 28); and f) a CDR-L3 having the amino acid sequence HQYLSSYT (SEQ ID NO: 29). In a particular embodiment, the antibody includes 6 CDRs with the following sequences: a) a CDR-H1 having the amino acid sequence GYTXTAYGIN (SEQ ID NO: 39); b) a CDR-H2 having the amino acid sequence YIYIGNGYTDYNEKFKG (SEQ ID NO: 40); c) a CDR-H3 having the amino acid sequence SGWDEDYAMDF (SEQ ID NO: 41); d) a CDR-L1 having the amino acid sequence RASENIYSYLA (SEQ ID NO: 42); e) a CDR-L2 having the amino acid sequence HAKTLAE (SEQ ID NO: 43); and f) a CDR-L3 having the amino acid sequence QHHYGPPPT (SEQ ID NO: 44). In a particular embodiment, the antibody or fragment inhibits an activity of human properdin.

In one embodiment, the disclosure is directed to use of an isolated monovalent antibody or antibody fragment thereof that binds human properdin in a method of treating a disease mediated by alternative complement pathway dysregulation or in the manufacture of a medicament for treating a disease mediated by alternative complement pathway dysregulation.

In one embodiment, the disclosure is directed to a method of treating a disease mediated by alternative complement pathway dysregulation. The methods includes administering an effective amount of the antibody of an isolated monovalent antibody or antibody fragment thereof, where the antibody or antibody fragment binds human properdin to a patient in need thereof. In a particular embodiment, the disease is autoimmune thrombotic thrombocytopenia purpura (TTP), hemolytic uremic syndrome (HUS), atypical hemolytic uremic syndrome (aHUS), paroxysmal nocturnal hemoglobinuria (PNH), IgA nephropathy (Berger's disease), asthma (e.g., severe asthma), C3 glomerulopathy (C3G), Gaucher disease, Hidradentitis suppurativa, Behcet's disease, severe burn, early sepsis, dermatomyositis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMD), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, membranoproliferative (MP) glomerulonephritis, membranous nephritism, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

In one embodiment, the disclosure is directed to a method of inhibiting alternative complement pathway membrane attack complex assembly. The method includes administering an effective amount of an antibody, antibody derivative or fragment thereof to a patient in need thereof. In a particular embodiment, the method inhibits alternative complement pathway dependent hemolysis.

In some embodiments, the antibody or fragment thereof includes the sequence of:

(SEQ ID NO: 45)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGSGGGGSGGGGSLE
VQLVESGGGLVQAGGSLRLSCAASGRISSIIHMAV
VYRQAPGKQRELVAEISRVGTTVYADSVKGRFTIS
RDDAKNTVTLQMNSLKPEDTAVYYCNALQYEKHGG
ADYWGQGTQVTVSS.

In some embodiments, the antibody or fragment thereof includes the sequence of:

(SEQ ID NO: 46)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGSGGGGSGGGGSEV
QLLESGGGLVQPGGSLRLSCAASGRISSIIHMAWF
RQAPGKERELVSEISRVGTTVYADSVKGRFTISRD
NSKNTLYLQMNSLKPEDTAVYYCNALQYEKHGGAD
YWGQGTLVTVSS.

In some embodiments, the antibody or fragment thereof includes the sequence of:

(SEQ ID NO: 47)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGDGGGGDGGGGEVQ
LVESGGGLVQAGGSLRLSCAASGRISSIIHMAVVY
RQAPGKQRELVAEISRVGTTVYADSVKGRFTISRD
DAKNTVTLQMNSLKPEDTAVYYCNALQYEKHGGAD
YWGQGTQVTVSS.

In some embodiments, the antibody or fragment thereof includes the sequence of:

(SEQ ID NO: 48)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGEGGGGEGGGGEVQ
LVESGGGLVQAGGSLRLSCAASGRISSIIHMAVVY
RQAPGKQRELVAEISRVGTTVYADSVKGRFTISRD
DAKNTVTLQMNSLKPEDTAVYYCNALQYEKHGGAD
YWGQGTQVTVSS.

In some embodiments, the antibody or fragment thereof includes the sequence of:

(SEQ ID NO: 49)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGSGGGGSGGGGSEV
QLVESGGGLVQPGGSLRLSCAASGRISSIIHMAVV
VRQAPGKQRELVSEISRVGTTVYADSVKGRFTISR
DNSKNTLYLQMNSLRAEDTAVYYCNALQYEKHGGA
DYWGQGTLVTVSS.

In some embodiments, the antibody or fragment thereof includes the sequence of:

(SEQ ID NO: 50)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGDGGGGDGGGGEVQ
LLESGGGLVQPGGSLRLSCAASGRISSIIHMAWFR
QAPGKERELVSEISRVGTTVYADSVKGRFTISRDN
SKNTLYLQMNSLKPEDTAVYYCNALQYEKHGGADY
WGQGTLVTVSS.

In some embodiments, the antibody or fragment thereof includes the sequence of:

(SEQ ID NO: 51)
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSGGGGEGGGGEGGGGEVQ
LLESGGGLVQPGGSLRLSCAASGRISSIIHMAWFR
QAPGKERELVSEISRVGTTVYADSVKGRFTISRDN
SKNTLYLQMNSLKPEDTAVYYCNALQYEKHGGADY
WGQGTLVTVSS.

In some embodiments, the antibody or fragment thereof includes the sequence of:

(SEQ ID NO: 52)
EVQLVESGGGLVKPGGSLRLSCAASGRPVSNYAAAWFRQAPGKEREFVS
AINWQKTATYADSVKGRFTISRDNAKNSLYLQMNSLRAEDTAVYYCAAV
FRVVAPKTQYDYDYWGQGTLVTVSSGGGGDGGGGDGGGGEVQLVESGGG
LVQPGGSLRLSCAASGRISSIIHMAWVRQAPGKQRELVSEISRVGTTVY
ADSVKGRFTISRDNSKNTLYLQMNSLRAEDTAVYYCNALQYEKHGGADY
WGQGTLVTVSS.

In some embodiments, the antibody or fragment thereof includes the sequence of:

(SEQ ID NO: 53)
LEVQLVESGGGLVQAGGSLRLSCAASGRISSIIHMAWYRQAPGKQRELV
AEISRVGTTVYADSVKGRFTISRDDAKNTVTLQMNSLKPEDTAVYYCNA
LQYEKHGGADYWGQGTQVTVSSRKCCVECPPCPAPPVAGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK.

In some embodiments, the antibody or fragment thereof is an LVP058 anti-properdin monovalent antibody VHH linked to an hG1 without a C1q binding domain and includes the sequence of:

(SEQ ID NO: 54)
LEVQLVESGGGLVQAGGSLRLSCAASGRISSIIHMAWYRQAPGKQRELV
AEISRVGTTVYADSVKGRFTISRDDAKNTVTLQMNSLKPEDTAVYYCNA
LQYEKHGGADYWGQGTQVTVSSPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK.

In some embodiments, the antibody or fragment thereof is an LVP058 anti-properdin monovalent antibody V_{HH} linked to an anti-albumin V_{HH} by a (G4S)₃ linker and includes the sequence of:

(SEQ ID NO: 55)
LEVQLVESGGGLVQAGGSLRLSCAASGRISSIIHMAWYRQAPGKQRELV
AEISRVGTTVYADSVKGRFTISRDDAKNTVTLQMNSLKPEDTAVYYCNA
LQYEKHGGADYWGQGTQVTVSSGGGGSGGGGSGGGGSEVQLVESGGGLV
KPGGSLRLSCAASGRPVSNYAAAWFRQAPGKEREFVSAINWQKTATYAD
SVKGRFTISRDNAKNSLYLQMNSLRAEDTAVYYCAAVFRVVAPKTQYDY
DYWGQGTLVTVSS.

### Definitions

As used herein, the term “monovalent antibody or antibody fragment thereof” refers to an antibody or antigen binding fragment thereof comprising a single binding domain, e.g., V_{H} or V_{HH}, for an antigen, e.g., a single properdin molecule. In one embodiment, the bound antigen molecule is part of a multimer, e.g., a trimer or higher order multimer of properdin monomers. Antibodies generally, including monovalent antibodies or antibody fragments thereof, bind with a high degree of specificity to a particular antigen.

As used herein, the term “single domain antibody” defines molecules where the antigen binding site is present on, and formed by, a single immunoglobulin domain. Generally, the antigen binding site of an immunoglobulin single variable domain is formed by no more than three CDRs. The single variable domain may, for example, include a light chain variable domain sequence (a V_{L} sequence) or a suitable fragment thereof; or a heavy chain variable domain sequence (e.g., a V_{H} sequence or V_{HH} sequence) or a suitable fragment thereof; as long as it is capable of forming a single antigen binding unit (i.e., a functional antigen binding unit that essentially is the single variable domain, such that the single antigen binding domain does not need to interact with another variable domain to form a functional antigen binding unit).

As used herein, the term “camelid antibody” refers to an antibody derived from a camelid species, for example, in a camel, dromedary, llama, alpaca or guanaco. Camelid antibodies differ from those of most other mammals in that they lack a light chain, and thus include only heavy chains with complete and diverse antigen binding capabilities (Hamers-Casterman, C. et al., Nature, 363:446-8, 1993).

As used herein, the term “V_{HH}” refers to a single heavy chain variable domain antibody devoid of light chains. V_{HH} chains, for example, can be of the type that can be found in Camelidae or cartilaginous fish that are naturally devoid of light chains or to a synthetic and non-immunized V_{HH} that can be constructed accordingly. Each heavy chain includes a variable region encoded by V-, D- and J-exons. A V_{HH} may be a natural V_{HH} antibody, e.g., a camelid antibody, or a recombinant protein including a heavy chain variable domain.

As used herein, the term an “isolated antibody” refers to an antibody that is substantially free of other antibodies having different antigenic specificities (e.g., an isolated antibody that binds to properdin is substantially free of contaminants, e.g., antibodies that do not bind to properdin). In addition, an “isolated” antibody is one that has been identified and separated and/or recovered from a component of its natural environment. Contaminant components of its natural environment are materials that could interfere with diagnostic or therapeutic uses for the antibody, and may include enzymes, hormones, and other proteinaceous or nonproteinaceous solutes.

As used herein, the term “specific binding” of an antibody or fragment thereof, polypeptide, or peptidomimetic is binding to a target molecule that is measurably different from binding to molecules that are not target molecules. As used herein, specific binding refers to a greater than 95% preference for binding a particular antigen versus background (“non-specific”) binding. “Substantially specific” binding refers to a greater than about 80% preference for binding a particular antigen versus background. Binding can be measured using a variety of methods including, but not limited to, Western blot, immunoblot, enzyme-linked immunosorbant assay (“ELISA”), radioimmunoassay (“RIA”), immunoprecipitation, surface plasmon resonance, bio-layer interferometry, chemiluminescence, fluorescent polarization, phosphorescence, immunohistochemical analysis, matrix-assisted laser desorption/ionization time-of-flight (“MALDI-TOF”) mass spectrometry, microcytometry, microarray, microscopy, fluorescence activated cell sorting (“FACS”) and flow cytometry.

As used herein, the term “human properdin” refers to a 469 amino acid soluble glycoprotein found in plasma that has seven thrombospondin type I repeats (TSR) with the N-terminal domain, TSR0, being a truncated domain. Human properdin, a 53 kDa protein, includes a signal peptide (amino acids 1-28), and six, non-identical TSR repeats about 60 amino acids each, as follows: amino acids 80-134 (TSR1), amino acids 139-191 (TSR2), amino acids 196-255 (TSR3), amino acids 260-313 (TSR4), amino acids 318-377 (TSR5), and amino acids 382-462 (TSR6). Properdin is formed by oligomerization of a rod-like monomer into cyclic dimers, trimers, and tetramers. The amino acid sequence of human properdin is found in the GenBank database under the following accession numbers: for human properdin, see, e.g., GenBank Accession Nos. AAA36489, NP-002612, AAH15756, AAP43692, S29126 and CAA40914. Properdin is a positive regulator of the alternative complement activation cascade. Known binding ligands for properdin include C3b, C3bB and C3bBb (Blatt, A. et al., Immunol. Rev., 274:172-90, 2016).

As used herein, the term “mouse properdin” refers to a 457 amino acid soluble glycoprotein found in plasma that has seven TSRs with the N-terminal domain, TSR0, being truncated. Mouse properdin, a 50 kDa protein, includes a signal peptide (amino acids 1-24), and six, non-identical TSRs of about 60 amino acids each, as follows: amino acids 73-130 (TSR1), amino acids 132-187 (TSR2), amino acids 189-251 (TSR3), amino acids 253-309 (TSR4), amino acids 311-372 (TSR5), and amino acids 374-457 (TSR6). Mouse properdin is formed by oligomerization of a rod-like monomer into cyclic dimers, trimers, and tetramers. The amino acid sequence of mouse properdin is found, for example, in the GenBank database (GenBank Accession Nos. P11680 and S05478).

As used herein, the term “TSR0 domain” refers to the truncated domain of properdin that precedes the TSR1 domain of properdin. For example, the TSR0 domain of human properdin includes amino acids 28-76.

As used herein, the term “TSR1 domain” refers to the domain of properdin adjacent to the TSR0 domain of properdin. For example, the TSR0 domain of human properdin includes amino acids 77-134.

As used herein, the term “an activity of properdin” refers to the biological activity of properdin including, but not limited to, binding interactions that lead to the stability of the C3/C5 convertase. Properdin binds most avidly to C3b, Bb—the alternative pathway C3/C5 convertase, but also binds to C3b; C3b, B and C3b, Bb. One function is to stabilize the C3b, Bb complex allowing increased alternative pathway activation (Pangburn, M., Methods Enzymol., 162:639-53, 1988; Nolan, K. & Reid, K., Methods Enzymol., 223:35-46, 1993). Properdin enhances formation of the alternative pathway C3 convertase by increasing binding of factor B to P,C3b complexes. Thus, properdin is an accelerator (positive regulator) of complement activation. Properdin also has been implicated in initiating activation of the alternative pathway by binding to the target surface and initiating C3/C5 convertase formation (Kemper C. & Hourcade, D., Mol. Immunol., 45:4048-56, 2008).

As used herein, the term “alternative complement pathway” refers to one of three pathways of complement activation (the others being the classical pathway and the lectin pathway). The alternative complement pathway is typically activated by bacteria, parasites, viruses or fungi, although IgA Abs and certain IgL chains have also been reported to activate this pathway.

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur both in fluid phase as well as at cell surface and can lead to excessive complement activation or insufficient regulation, both causing tissue injury.

As used herein, the term “a disease mediated by alternative complement pathway dysregulation” refers to an interruption, cessation or disorder of body functions, systems or organs caused by alternative complement pathway dysregulation. Such diseases would benefit from treatment with a composition or formulation described herein. In some embodiments, the disease is caused by any aberration in the ability of the alternative complement pathway to provide host defense against pathogens and clear immune complexes and damaged cells, and for immunoregulation. Also encompassed herein are diseases, directly or indirectly, mediated by dysregulation of one or more components of the alternative complement pathway, or a product generated by the alternative complement pathway.

As used herein, the term “alternative complement pathway-dependent membrane attack complex assembly” refers to a terminal complex formed as a result of alternative complement pathway activation and includes complement components C5, C6, C7, C8 and C9. Assembly of the membrane attack complex (MAC) leads to cell lysis.

As used herein, the term “alternative complement pathway dependent hemolysis” refers to the lysis of red blood cells mediated by increased alternative complement pathway-dependent MAC assembly and/or deposition on red blood cells.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. A linker may refer to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains. An amino acid spacer may be part of the primary sequence of a polypeptide (e.g., joined to the spaced polypeptides or polypeptide domains via the polypeptide backbone). A linker may comprise one or more glycine and serine residues.

### BRIEF DESCRIPTION OF THE DRAWINGS

FIG. 1 depicts bio-layer interferometry data obtained using an Octet™ biosensor with a model system in which selected anti-properdin antibodies specifically bind human properdin. The graph shows equilibrium dissociation over time.

FIG. 2 depicts bio-layer interferometry data obtained using an Octet™ biosensor with a model system in which selected anti-properdin antibodies showed weak or no binding to mouse properdin. The graph shows equilibrium dissociation over time.

FIG. 3 depicts bio-layer interferometry data obtained using an Octet™ biosensor with a model system in which selected anti-properdin antibodies showed specific, but weak binding to cynomolgus properdin. The graph shows equilibrium dissociation over time.

FIG. 4 shows that selected anti-properdin antibodies inhibit activity of human properdin in an alternative complement pathway hemolysis assay.

FIG. 5A to FIG. 5C show the characterization of selected anti-properdin antibodies by mass spectrometry.

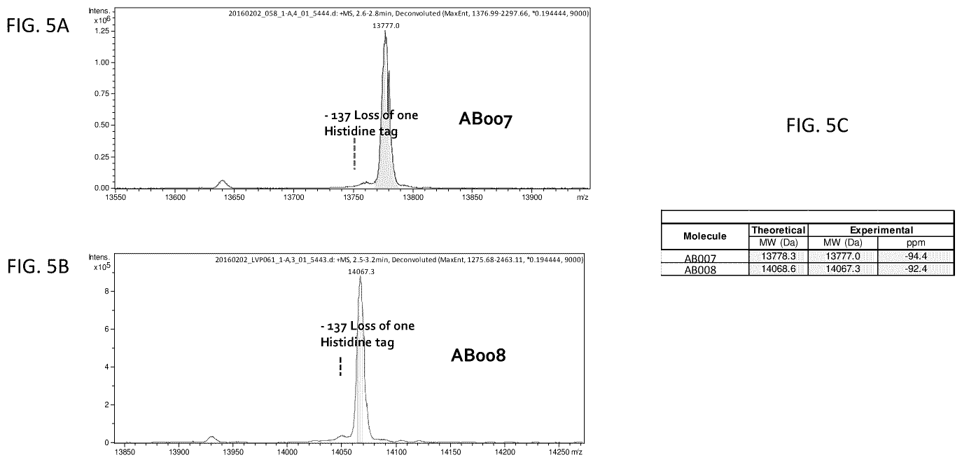

FIG. 6A and FIG. 6B show the binding affinity of selected anti-properdin antibodies to biotinylated properdin using a properdin capture method.

FIG. 7 shows the binding affinity of selected anti-properdin bispecific antibodies to biotinylated properdin using a properdin capture method.

FIG. 8A and FIG. 8B show selected anti-properdin bispecific antibodies inhibit activity of human and cynomolgus properdin in an alternative complement pathway hemolysis assay. An anti-properdin antibody was used as the control.

FIG. 9A and FIG. 9B show selected anti-properdin bispecific antibodies inhibit activity of human and cynomolgus properdin in an alternative complement pathway hemolysis assay.

FIG. 10A and FIG. 10B show the binding affinity of selected anti-properdin bispecific antibodies to biotinylated properdin using a properdin capture method.

FIG. 11A and FIG. 11B show the binding affinity of selected anti-properdin bispecific antibodies to biotinylated properdin using a properdin capture method.

FIG. 12A and FIG. 12B show the binding affinity of selected anti-properdin bispecific antibodies to biotinylated properdin using a properdin capture method.

### DETAILED DESCRIPTION

Properdin is a positive regulator of the alternative complement pathway. Described herein are novel monovalent antibodies that bind to a single properdin molecule and are useful for treating diseases mediated by dysregulation of the alternative complement pathway. Described herein is the discovery that immune complexes resulting from bivalent antibodies binding more than one properdin multimer exhibit toxicity as therapeutic agents for inhibiting aberrant activation of the alternative complement pathway. Monovalent antibodies described herein have a 1:1 binding ratio to properdin and, by design, cannot form antibody/properdin aggregates containing more than one properdin multimer, providing an advantage over bivalent and multivalent antibodies.

The sections that follow provide a description of monovalent antibodies or antibody fragments that can be administered to a patient with disease mediated by alternative complement pathway dysregulation.

### Anti-Properdin Antibodies

Described herein are monovalent anti-properdin antibodies, antibody derivatives (e.g., engineered antibodies, humaneered antibodies, chimeric antibodies, substituted antibodies, humanized antibodies etc.) and antibody fragments thereof that inhibit properdin, a positive regulator of the alternate pathway of complement, and subsequently destabilize the C3- and C5-convertase enzyme complexes. The antibodies described herein can inhibit, for example, properdin binding to C3b, C3Bb, and C3bBb. Inhibition of properdin leads to reduced alternative pathway complement activation, indicating a therapeutic benefit for patients afflicted with a disease of alternative pathway dysregulation wherein the alternative pathway is hyper-activated.

Anti-properdin antibodies described herein can be produced by using full-length properdin, properdin polypeptides, and/or using antigenic properdin epitope-bearing peptides, for example, a fragment of the properdin polypeptide. Properdin peptides and polypeptides can be isolated and used to generate antibodies as natural polypeptides, recombinant or synthetic recombinant polypeptides. All antigens useful for producing anti-properdin antibodies can be used to generate monovalent antibodies. Suitable monovalent antibody formats, and methods for producing them, are known in the art (e.g., WO 2007/048037 and WO 2007/059782, the entire contents of which are incorporated herein by reference).

The anti-properdin antibody may be a monoclonal antibody or derived from a monoclonal antibody. Suitable monoclonal antibodies to selected antigens may be prepared by known techniques (“Monoclonal Antibodies: A manual of techniques,” Zola (CRC Press, 1988); “Monoclonal Hybridoma Antibodies: Techniques and Applications,” Hurrell (CRC Press, 1982), the entire contents of which are incorporated herein by reference).

In other embodiments, the antibody may be a single-domain antibody, such as a V_{HH}. Such antibodies exist naturally in camelids and sharks (Saerens, D. et al., Curr. Opin. Pharmacol., 8:600-8, 2008). Camelid antibodies are described in, for example, U.S. Pat. Nos. 5,759,808; 5,800,988; 5,840,526; 5,874,541; 6,005,079; and 6,015,695, the entire contents of each of which are incorporated herein by reference. The cloned and isolated V_{HH} domain is a stable polypeptide that features the full antigen-binding capacity of the original heavy-chain antibody. V_{HH} domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanized (U.S. Pat. Nos. 5,840,526; 5,874,541; 6,005,079, 6,765,087; EP 1589107; WO 97/34103; WO 97/49805; U.S. Pat. Nos. 5,800,988; 5,874,541 and 6,015,695, the entire contents of each of which are incorporated herein by reference).

In some embodiments, the antibody or antibody fragment thereof includes the following sequence as its heavy chain variable domain:

(SEQ ID NO: 71)
QVQLVESGGGLVQAGGSLRLSCAASGRTFSSYAMGWFRQAPGKEREFVA
AIGWNGEGIYYADSVKGRFTISRDNAKNTGYLQMNSLKPEDTAVYYCAA
DSEGVVPGFPIAYWGQGTQVTVSG

In some embodiments, the antibody or antibody fragment thereof includes the following sequence as its heavy chain variable domain:

(SEQ ID NO: 72)
QVQLVESGGGLVQPGGSLRLSCAASGFPLNSYAIGWFRQAPGKEREGVS
CISVSDDSTYYTDSVKGRFTISRDNAKNTVYLQMNSLKPEDTAVYYCAV
DSAPLYGDYVCKPLENEYDFWGQGTQVTVSG.

In some embodiments, the antibody or antibody fragment thereof includes the following sequence as its heavy chain variable domain:

(SEQ ID NO: 73)
QVQLVESGGGLVQAGGSLXLSCAASGSDRRINGMGWYRHPPGKQRELVA
AITSGGSTNYADSVKGRFTISTNNANNMMYLQMNSLKPEDTAVYYCAID
EFGTGWLDYCGQGTQVTVSG.

In some embodiments, the antibody or antibody fragment thereof includes the following sequence as its heavy chain variable domain:

(SEQ ID NO: 74)
QVQLVESGGGLVQPGGSLRLSCAASGRPFSSYAMGWFRQAPGKEREIVA
GLSWSGGNVYYADSVKGRFTISRDNAKNTGDLQMNSLKPEDTAVYYCAI
GPKLTTGPTAYRYWGQGTQVTVSS

In some embodiments, the antibody or antibody fragment thereof includes the following sequence as its heavy chain variable domain:

(SEQ ID NO: 75)
QVQLVESGGGLVQPGGSLRLSCATSGGTFSSYAMGWFRQAPGKEREFVA
AITWNGSNRYYADSVKGRFTISRDNAKSTVYLQMNSLKPEDTAVYYCAA
EHSTRYSGFYYYTRGETYHYWGQGTQVTVSG

In some embodiments, the antibody or antibody fragment thereof includes the following sequence as its heavy chain variable domain:

(SEQ ID NO: 76)
QVQLVESGGGLVQAGGSLRLSCAASGRTFSTLGMGWFRQAPGKERQFVA
AINWSGSSTYYANSVKGRFTISRDNAQSTMYLQMNSLKPEDTAVYYCAA
DLDSRYSAYYYYSDESQYDYWGQGTLVTVSG

In some embodiments, the antibody or antibody fragment thereof includes the following sequence as its heavy chain variable domain:

(SEQ ID NO: 77)
QVQVVESGGGLVQPGGSLSLSCAASGRTFSSYAMGWFRQAPGKEREFVA
AITWDGANIYYADSVKGRFTLSRDNAENTVWLQLNSLKPEDTAVYYCAA
AESGRYSGRDYYSAPGVYLYWGQGTLVTVSG

In some embodiments, the antibody or antibody fragment thereof includes the following sequence as its heavy chain variable domain:

(SEQ ID NO: 78)
QVQLVESGGGLVQAGGSLRLSCAASGSIFDINAMGWYRQAPGKQRELVA
DITSSGSTNYADSVKGRFTISRDNAKNTVYLQMNSLKPEDTAVYTCAAE
SIRESQNRHQLGYMGPLYDYWGQGTQVTVSG

In some embodiments, the antibody or antibody fragment thereof includes the following sequence as its heavy chain variable domain:

(SEQ ID NO: 79)
QVQLIESGGGLVQAGDSLRLSCAASEGTFSRFAMGWFRQAPGKEREFVA
AINWSGGITYYADSVKGRFTISRDNAKNTVYLQMNSLKPEDTADYYCAA
ETTTRYSGYYYYEDNKSYDYWGQGTLVTVSG

In some embodiments, the antibody or antibody fragment thereof includes the following sequence as its heavy chain variable domain:

(SEQ ID NO: 58)
QVQVVESGGGLRQTGGSLRLSCTASGRIFEVNMMAWYRQAPGKQRELVA
EISRVGTTVYADSVKGRFTISRDSAKNTVTLQMNSLKSEDTAVYYCNAL
QYDRYGGAEYWGQGTQVTVSS

In some embodiments, the antibody or antibody fragment thereof includes the following sequence as its heavy chain variable domain:

(SEQ ID NO: 80)
QVLLEESGGGLERTGGSLRLSCAASGSIFSVNSMTWYRQAPGKRREFLG
TITEEGRTNYADSVKGRFTISRDNAKNTMYLQMNSLKPEDTAVYYCYAN
LISSEDRTFGVWGQGTQVTVSS

In some embodiments, the antibody or antibody fragment thereof includes the following sequence as its heavy chain variable domain:

(SEQ ID NO: 81)
QVHLVESGGGLVQAGGSLRLSCTASGGTVGDYAVGWFRQAPGKERELIG
VVSRLGARTGYADSVLGRFTISRDDVKNTVFLQMDSVKPEDTAVYYCAA
RRDYSFEVVPYDYWGQGTQVTVSG

In some embodiments, the antibody or antibody fragment thereof includes the following sequence as its heavy chain variable domain:

(SEQ ID NO: 82)
QVQMVESGGGLVQAGGSLRLSCAASGLTNRIRIMGWYRQAPGKLRELVA
TITNDGSTHYADSVKGRFTISTDNAKNTVFLQMNSLKPEDTAVYICNVG
ENWGPAYWGQGTQVTVSG

In some embodiments, the antibody or antibody fragment thereof includes the following sequence as its heavy chain variable domain:

(SEQ ID NO: 72)
QVQLVESGGGLVQPGGSLRLSCAASGFPLNSYAIGWFRQAPGKEREGVS
CISVSDDSTYYTDSVKGRFTISRDNAKNTVYLQMNSLKPEDTAVYYCAV
DSAPLYGDYVCKPLENEYDFWGQGTQVTVSG

In some embodiments, the antibody or antibody fragment thereof includes the following sequence as its heavy chain variable domain:

(SEQ ID NO: 83)
QVRLTESGGGLVQYGTNLTLTCVASGLISTRNKMGWFRRRSGGQREFVA
SSTVLSDDVIQDDIAETVKGRFAVARNDYKNILYLQMTAVKPEDTGFYW
CASGTSLFGASRREDDFNAWGVGTQVTVSA

In some embodiments, the antibody or antibody fragment thereof includes the following sequence as its heavy chain variable domain:

(SEQ ID NO: 59)
QVQLAESGGGLVQAGDSLKLSCTASGRIFEVNMMAWYRQAPGKDRELVA
EISRVGTTTYADSVKGRFTISRDSAKNTVTLQMNSLKSEDTAVYYCNAL
QYSRYGGAEYWGQGTQVTVSG

In some embodiments, the antibody or antibody fragment thereof includes the following sequence as its heavy chain variable domain:

(SEQ ID NO: 84)
EVQLVESGGGLVQPGGSLRLSCAASGFTFGSADMSWVRQAPGKGPEWVS
AINSNGGSTYYAASVKGRFTISRDNAKNTLYLQMNSLKPEDTAVYYCAQ
GNWYTEEYHYWGQGTLVTVSG

In some embodiments, the antibody or antibody fragment thereof includes the following sequence as its heavy chain variable domain:

(SEQ ID NO: 85)
QVRLVESGGGLVQAGDSLRLSCAASGRTLSSYAMGWFRQAPGKEREFVA
ATTWRDTSTYYADSVKGRFTISRDNAKNTVYLQMNSLKPEDTAAYYCAA
EEPSKYSGRDYYMMGDSYDYWGQGTQVTVSS

In some embodiments, the antibody or antibody fragment thereof includes the following sequence as its heavy chain variable domain:

(SEQ ID NO: 86)
EVQLVESGGGLVQPGGSLRLSCAASGFTFGSADMSWVRQAPGKGPEWVS
AINSNGGSTYYAASVKGRFTISRDNAKNTLYLQMNSLKPEDTAVYYCAQ
GNWYTEEYHYWGQGTQVTVSG

In some embodiments, the antibody or antibody fragment thereof includes the following sequence as its heavy chain variable domain:

(SEQ ID NO: 87)
QVQLVESGGGLVQAGGSLRLSCAASGRTFSNYAMAWFRQAPGKEREFVA
SISGSGDSRYYADSVKGRFTISRDNAKNTVYLQTNSPKPEDTAVYYCAA
VLPTRYSGFYYYSDGTQYHYWGQGTQVTVSS

In some embodiments, the antibody or antibody fragment thereof includes the following sequence as its heavy chain variable domain:

(SEQ ID NO: 88)
QVNLVESGGGSVQAGGSLRLSCAASENINVINDMGWYRQAPGKQRELVA
VITGHDNINYADSATGRFTISTYTWNTENLQMNMLKPEDTAVYYCNADI
TYANGRFNDWGQGTQVTVSS

In some embodiments, the antibody or antibody fragment thereof includes the following sequence as its heavy chain variable domain:

(SEQ ID NO: 89)
QVHLVESGGGLVQAGGSLRLSCAASGRTFSSYAMGWFRQPPGKEREFVA
AITWSGSSIYYADSVKGRFTISRDNAKNTVYLQMNSLKPEDTAVYYCAA
EETSKYSGSYYYMMGDSYDYWGQGTQVTVSG

In some embodiments, the antibody or antibody fragment thereof includes the following sequence as its heavy chain variable domain:

(SEQ ID NO: 90)
QVQLVESGGGLVQAGGSLRLSCAASGRTFSSYAMGWFRQAPGKEREFVA
AVPWTYGSKYYADSVKGRFTISRDDAKNTVYLQMNNLKPEDTAVYYCAA
DSSAGYYSGFDYYSAATPYDLWGQGTQVTVSG

In some embodiments, the antibody or antibody fragment thereof includes the following sequence as its heavy chain variable domain:

(SEQ ID NO: 91)
QVQLVESGGGLVQPGGSLRLSCAASGSDYYAIGWFRQAPGKEREGVSCM
SRTDGSTYYADSVKDRFTISRDYAKNTVYLQMNSLKPEDTAVYYCGLDR
SYPTGGISCLFGDFGSWGQGTQVTVSG

In some embodiments, the antibody or antibody fragment thereof includes the following sequence as its heavy chain variable domain:

(SEQ ID NO: 92)
QVQLVESGGGLVQAGGSLRLSCAASGRTFSSYNMGWFRQRHGNEREFVA
TISWSGRSTYYADSVKGRFAISRDNANTTVYLQMNSLKPEDSAVYYCAA
STRGWYGTQEDDYNFWGQGTQVTVSG

In some embodiments, the antibody or antibody fragment thereof includes the following sequence as its heavy chain variable domain:

(SEQ ID NO: 60)
QVQLVESGGGLVQAGGSLRLSCAASGRISSIIHMAWYRQAPGKQRELVAE
ISRVGTTVYADSVKGRFTISRDDAKNTVTLQMNSLKPEDTAVYYCNALQY
EKHGGADYWGQGTQVTVSG

In some embodiments, the antibody or antibody fragment thereof includes the following sequence as its heavy chain variable domain:

(SEQ ID NO: 93)
QVQLVESGGGLVQAGGSLRLSCAASGGTFSSYSMGWFRQAPGKEREFVAA
ITWNGVSTYYADSVKGRFTISRDNAKNTVYLQMNSLKPTDTAVYYCAAEI
TTRYSGFYYYEDNKSYDYWGQGTQVTVSS

In some embodiments, the antibody or antibody fragment thereof includes the following sequence as its heavy chain variable domain:

(SEQ ID NO: 61)
QVQLVESGGGLRQTGESLRLSCTASGRIFEVNMMAWYRQAPGKQRELVAE
ISRVGTTTYADSVKGRFTISRDSAKNTVTLQMNSLKSEDTAVYYCNALQY
DRYGGAEYWGQGTQVTVSG

In some embodiments, the antibody or antibody fragment thereof includes the following sequence as its heavy chain variable domain:

(SEQ ID NO: 94)
EVQLVESGGGLVQPGGSLRLSCAASGFTLDYYAIGWFRQAPGKEREGVSC
ISRTDGSTYYADSVKGRFTISRDNAKNTVYLQMNSLKPEDTAVYYCAVDD
SYPTGGISCLFGHFGSWGQGTQVTVSS

In some embodiments, the antibody or antibody fragment thereof includes the following sequence as its heavy chain variable domain:

(SEQ ID NO: 95)
QVQLVESGGGLVQAGDSLRLSCAASGFTFSSYAMGWFRQAPGKEREFVAA
ITWSGVSTYYADSVKGRFTISRDNAKNRVYLQMNSLKPEDTAVYSCAADG
SGRYSGMEYYNRDWVYDYWGQGTQVTVSS

In some embodiments, the antibody or antibody fragment thereof includes the following sequence as its heavy chain variable domain:

(SEQ ID NO: 96)
QVHMVESGGGLVQAGGSLRFSCAASGNIFTISTLDWYRQAPGEQRELVAT
LTPDGITDYAGSVKGRFTISRDNAKNTVYLQMNSLKPEDTAVYYCNAWRY
SDDYRGRVDYWGQGTQVTVSG

In some embodiments, the antibody or antibody fragment thereof includes the following sequence as its heavy chain variable domain:

(SEQ ID NO: 97)
QVQLIESGGGLVQEGASLRLSCAGSGPMFSRLAVGWFRQAPGKEREFVAV
INWSGSADFYTNSVKGRFTISRDNAKNTVYLEMNTLKPEDSAVYYCAADQ
NPLTLRTGVRDVGRQWGQGTEVTVSS

In some embodiments, the antibody or antibody fragment thereof includes the following sequence as its heavy chain variable domain:

(SEQ ID NO: 98)
QVQLVESGGGLVQAGGSLRLSCAASGRTFSSYAMGWFRQAPGKEREFVAA
ITWRGASTYYADPVKGRFTISRDNAKNTVYLQMSSLKPEDTAVYYCAAEE
PSYYSGSYYYMMGDSYNYWGQGTQVTVSG

In some embodiments, the antibody or antibody fragment thereof includes the following sequence as its heavy chain variable domain:

(SEQ ID NO: 99)
QVQLVESGGGLVQAGGSLRLSCTASGRTFSNYAMGWFRQAPGKEREFLAA
ISRSGESTNYATFVKGRFTIARDNAKNTVSLQMNSLKPEDTAVYFCAAKV
AVLVSTTYNSQYDYWGQGTQVTVSS.

Anti-properdin antibodies, antibody derivatives and fragments thereof disclosed herein include those that have one or more, or all, of the following CDRs:
a. a CDR-H1 having the amino acid sequence GRIFEVNMMA (SEQ ID NO: 9);
b. a CDR-H2 having the amino acid sequence RVGTTVYADSVKG (SEQ ID NO: 12);
c. a CDR-H3 having the amino acid sequence LQYDRYGGAEY (SEQ ID NO: 13).

Additional anti-properdin antibodies, antibody derivatives and fragments thereof disclosed herein include those that have one or more, or all, of the following CDRs:
a. a CDR-H1 having the amino acid sequence GRIFEVNMMA (SEQ ID NO: 9);
b. a CDR-H2 having the amino acid sequence RVGTTTYADSVKG (SEQ ID NO: 15); and
c. a CDR-H3 having the amino acid sequence LQYSRYGGAEY (SEQ ID NO: 14).

Additional anti-properdin antibodies, antibody derivatives and fragments thereof disclosed herein include those that have one or more, or all, of the following CDRs:
a. a CDR-H1 having the amino acid sequence GRIFEVNMMA (SEQ ID NO: 9);
b. a CDR-H2 having the amino acid sequence RVGTTTYADSVKG (SEQ ID NO: 15); and
c. a CDR-H3 having the amino acid sequence LQYDRYGGAEY (SEQ ID NO: 13).

Additional anti-properdin antibodies, antibody derivatives and fragments thereof disclosed herein include those that have one or more, or all, of the following CDRs:
a. a CDR-H1 having the amino acid sequence GRISSIIHMA (SEQ ID NO: 16);
b. a CDR-H2 having the amino acid sequence RVGTTVYADSVKG (SEQ ID NO: 12); and
c. a CDR-H3 having the amino acid sequence LQYEKHGGADY (SEQ ID NO: 17).

Humanized camelid V_{HH} polypeptides are taught, for example in WO04/041862, the teachings of which are incorporated herein in their entirety. It will be understood by one of skill in the art that naturally occurring camelid antibody single variable domain polypeptides can be modified (e.g., amino acid. substitutions at positions 45 and 103 (WO04/041862)) to generate humanized camelid V_{HH} polypeptides. Also included herein are antibody single variable domain polypeptides that are nurse shark V_{HH} (Greenberg, A. et al., Nature, 374:168-73, 1995; U.S. Patent Publication No. 20050043519).

Additional anti-properdin antibodies, antibody derivatives and fragments thereof disclosed herein include those that have one or more, or all (e.g., to create a scFv or dAb), of the following CDRs:
a) a CDR-H1 having the amino acid sequence GYIFTNYPIH (SEQ ID NO: 18);
b) a CDR-H2 having the amino acid sequence FIDPGGGYDEPDERFRD (SEQ ID NO: 19);
c) a CDR-H3 having the amino acid sequence RGGGYYLDY (SEQ ID NO: 20);
d) a CDR-L1 having the amino acid sequence RASQDISFFLN (SEQ ID NO: 21);
e) a CDR-L2 having the amino acid sequence YTSRYHS (SEQ ID NO: 22); and
f) a CDR-L3 having the amino acid sequence QHGNTLPWT (SEQ ID NO: 23).

Additional anti-properdin antibodies, antibody derivatives and fragments thereof disclosed herein include those that have one or more, or all (e.g., to create a scFv), of the following CDRs:
a) a CDR-H1 having the amino acid sequence GFSLTTYGVH (SEQ ID NO: 24);
b) a CDR-H2 having the amino acid sequence VIWSGGDTDYNASFIS (SEQ ID NO: 25);
c) a CDR-H3 having the amino acid sequence NKDYYTNYDFTMDY (SEQ ID NO: 26);
d) a CDR-L1 having the amino acid sequence KSSQSVLYSSNQKNFLA (SEQ ID NO: 27);
e) a CDR-L2 having the amino acid sequence WASTRES (SEQ ID NO: 28); and
f) a CDR-L3 having the amino acid sequence HQYLSSYT (SEQ ID NO: 29).

Additional anti-properdin antibodies, antibody derivatives and fragments thereof disclosed herein include those that have one or more, or all (e.g., to create a scFv), of the following CDRs:
a) a CDR-H1 having the amino acid sequence GYTFIDYWIE (SEQ ID NO: 30);
b) a CDR-H2 having the amino acid sequence EIFPGSGTINHNEKFKD (SEQ ID NO: 31);
c) a CDR-H3 having the amino acid sequence EGLDY (SEQ ID NO: 32);
d) a CDR-L1 having the amino acid sequence SASSSVSYIY (SEQ ID NO: 33);
e) a CDR-L2 having the amino acid sequence DTSTLAS (SEQ ID NO: 34); and
f) a CDR-L3 having the amino acid sequence QQWSRNPFT (SEQ ID NO: 35).

Additional anti-properdin antibodies, antibody derivatives and fragments thereof disclosed herein include those that have one or more, or all (e.g., to create a scFv), of the following CDRs:
a) a CDR-H1 having the amino acid sequence GFSLTSYGVH (SEQ ID NO: 36);
b) a CDR-H2 having the amino acid sequence VIWSGGSTDYNAAFIS (SEQ ID NO: 37);
c) a CDR-H3 having the amino acid sequence NKDFYSNYDYTMDY (SEQ ID NO: 38);
d) a CDR-L1 having the amino acid sequence KSSQSVLYSSNQKNFLA (SEQ ID NO: 27);
e) a CDR-L2 having the amino acid sequence WASTRES (SEQ ID NO: 28); and
f) a CDR-L3 having the amino acid sequence HQYLSSYT (SEQ ID NO: 29).

Additional anti-properdin antibodies, antibody derivatives and fragments thereof disclosed herein include those that have one or more, or all (e.g., to create a scFv), of the following CDRs:
a) a CDR-H1 having the amino acid sequence GYTXTAYGIN (SEQ ID NO: 39);
b) a CDR-H2 having the amino acid sequence YIYIGNGYTDYNEKFKG (SEQ ID NO: 40);
c) a CDR-H3 having the amino acid sequence SGWDEDYAMDF (SEQ ID NO: 41);
d) a CDR-L1 having the amino acid sequence RASENIYSYLA (SEQ ID NO: 42);
e) a CDR-L2 having the amino acid sequence HAKTLAE (SEQ ID NO: 43); and
f) a CDR-L3 having the amino acid sequence QHHYGPPPT (SEQ ID NO: 44).

In some embodiments, the antibody or antibody fragment thereof includes the following sequence:

(SEQ ID NO: 53)
LEVQLVESGGGLVQAGGSLRLSCAASGRISSIIHMAWYRQAPGKQRELVA
EISRVGTTVYADSVKGRFTISRDDAKNTVTLQMNSLKPEDTAVYYCNALQ
YEKHGGADYWGQGTQVTVSSRKCCVECPPCPAPPVAGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

In some embodiments, the antibody or antibody fragment thereof includes the following sequence:

(SEQ ID NO: 54)
LEVQLVESGGGLVQAGGSLRLSCAASGRISSIIHMAWYRQAPGKQRELVA
EISRVGTTVYADSVKGRFTISRDDAKNTVTLQMNSLKPEDTAVYYCNALQ
YEKHGGADYWGQGTQVTVSSPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDVVLNGKEYKCAVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK

In some embodiments, the antibody or antibody fragment thereof includes the following light chain and heavy chain sequences:

(SEQ ID NO: 56)
DIQMTQSPSSLSASVGDRVTITCRASQDISFFLNWYQQKPGKAPKLLIYY
TSRYHSGVPSRFSGSGSGTDFTLTISSLQPEDFATYYCQHGNTLPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;
and
(SEQ ID NO: 57)
QVQLVQSGAEVKKPGASVKVSCKASGYIFTNYPIHWVRQAPGQGLEWMGF
IDPGGGYDEPDERFRDRVTMTRDTSTSTVYMELSSLRSEDTAVYYCARRG
GGYYLDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKRVEPKSCDKTHTCPPCPAPELLGGPSVFLFPPKPKD
TLMISRTPEVTCVVVDVSHEDPEVKFNWYVDGVEVHNAKTKPREEQYNST
YRVVSVLTVLHQDWLNGKEYKCAVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK

In some embodiments, the antibody or antibody fragment thereof includes the following sequence:

(SEQ ID NO: 55)
LEVQLVESGGGLVQAGGSLRLSCAASGRISSIIHMAWYRQAPGKQRELVA
EISRVGTTVYADSVKGRFTISRDDAKNTVTLQMNSLKPEDTAVYYCNALQ
YEKHGGADYWGQGTQVTVSSGGGGSGGGGSGGGGSEVQLVESGGGLVKPG
GSLRLSCAASGRPVSNYAAAWFRQAPGKEREFVSAINWQKTATYADSVKG
RFTISRDNAKNSLYLQMNSLRAEDTAVYYCAAVFRVVAPKTQYDYDYWGQ
GTLVTVSS

In some embodiments, the antibody or antibody fragment thereof includes the following sequence:

(SEQ ID NO: 45)
EVQLVESGGGLVKPGGSLRLSCAASGRPVSNYAAAWFRQAPGKEREFVSA
INWQKTATYADSVKGRFTISRDNAKNSLYLQMNSLRAEDTAVYYCAAVFR
VVAPKTQYDYDYWGQGTLVTVSSGGGGSGGGGSGGGGSLEVQLVESGGGL
VQAGGSLRLSCAASGRISSIIHMAWYRQAPGKQRELVAEISRVGTTVYAD
SVKGRFTISRDDAKNTVTLQMNSLKPEDTAVYYCNALQYEKHGGADYWGQ
GTQVTVSS

### Anti-Properdin Antibody Fragments and Derivatives

Some naturally occurring antibodies include two antigen binding domains and are therefore divalent. A number of smaller antigen binding fragments of naturally occurring antibodies have been identified following protease digestion. These include, for example, the “Fab fragment” (V_{L}-C_{L}-C_{H}1-V_{H}), “Fab′ fragment” (a Fab with the heavy chain hinge region), and “F(ab′)₂ fragment” (a dimer of Fab′ fragments joined by the heavy chain hinge region). Recombinant methods have been used to generate such fragments and to generate even smaller antibody fragments, e.g., those referred to as “single chain Fv” (variable fragment) or “scFv,” consisting of V_{L} and V_{H} joined by a synthetic peptide linker (V_{L}-linker-V_{H}). Fab fragments, Fab′ fragments and scFv fragments are monovalent for antigen binding, as they each include only one antigen binding domain including one V_{H}/V_{L} dimer. Even smaller monovalent antibody fragments are the dAbs, which include only a single immunoglobulin variable domain, e.g., V_{H} or V_{L}, that alone specifically binds antigen, i.e., without the need for a complementary V_{L} or V_{H} domain, respectively. A dAb binds antigen independently of other V domains; however, a dAb can be present in a homo- or hetero-multimer with other V_{H} or V_{L} domains where the other domains are not required for antigen binding by the dAb, i.e., where the dAb binds antigen independently of the additional V_{H} or V_{L} domains.

### Linkers

In the present invention, a linker is used to describe a linkage or connection between polypeptides or protein domains and/or associated non-protein moieties. In some embodiments, a linker is a linkage or connection between at least two polypeptide constructs, e.g., such that the two polypeptide constructs are joined to each other in tandem series (e.g., a monovalent antibody linked to a second polypeptide or monovalent antibody). A linker can attach the N-terminus or C-terminus of one antibody construct to the N-terminus or C-terminus of a second polypeptide construct.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA polymerase and ribosome, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

In the present invention, a linker between two peptide constructs can be an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine. In certain embodiments, a linker can contain single motifs or multiple different or repeating motifs, of GS, GGS, GGGGS (SEQ ID NO: 1), GGSG (SEQ ID NO: 2), or SGGG (SEQ ID NO: 3). Exemplary motifs have the sequence of (G4S)ₙ, (G4D)ₙ, (G4E)ₙ, (G4A)ₙ where n=1, 2, 3, 4, 5, or more, and combinations thereof. Other linkers include the sequences GGGGD (SEQ ID NO: 63), GGGGE (SEQ ID NO: 64), and GGGGA (SEQ ID NO: 100). Linkers can be designed by combining these various motifs. Such linkers include

(SEQ ID NO: 4)
GGGGSGGGGSGGGGS,
(SEQ ID NO: 5)
GGGGDGGGGDGGGG,
(SEQ ID NO: 6)
GGGGEGGGGEGGGG,
and
(SEQ ID NO: 101)
GGGGAGGGGAGGGGS.

### Bispecific Constructs

The invention also features bispecific constructs where two antigen binding polypeptides are linked (e.g., by a linker such as the linker of any one of SEQ ID NOs: 1-6, 63-64, and 100-101). Such bispecific constructs may include an anti-properdin binding polypeptide (e.g., a monovalent antibody) connected by a linker to a second polypeptide (e.g., a second monovalent antibody). The second polypeptide can enhance in vivo stability of the bispecific construct. In some embodiments, the second polypeptide is an albumin binding molecule, an albumin binding peptide, or an anti-albumin antibody (e.g., a monovalent antibody), or a modified form thereof. Albumin binding peptides are known in the art and are described, for example, in WO 2007/106120 (see Tables 1 to 9) and Dennis et al., 2002, J Biol. Chem. 277:35035-35043, the disclosures of which are hereby incorporated by reference.

In some embodiments, the second polypeptide is a Fc domain that enhances in vivo stability of the construct.

Exemplary bispecific constructs are shown below in Example 5.

In some embodiments, a monovalent anti-properdin antibody is linked to a monovalent anti-albumin antibody. The monovalent anti-properdin antibody may be linked by its N-terminus or C-terminus to the N-terminus or C-terminus of the monovalent anti-albumin antibody.

The monovalent anti-properdin antibody may be linked by its N-terminus or C-terminus to the N-terminus or C-terminus of the monovalent anti-albumin antibody with a linker with the amino acid sequence of any one of SEQ ID NOs: 1-6, 63-64, and 100-101.

In some embodiments, a monovalent anti-properdin antibody including the amino acid sequence of SEQ ID NO: 58 is linked to a monovalent anti-albumin antibody. The monovalent anti-properdin antibody including the sequence of SEQ ID NO: 58 may be linked by its N-terminus or C-terminus to the N-terminus or C-terminus of the monovalent anti-albumin antibody with a linker including the amino acid sequence of any one of SEQ ID NOs: 1-6, 63-64, and 100-101.

In some embodiments, a monovalent anti-properdin antibody including the amino acid sequence of SEQ ID NO: 59 is linked to a monovalent anti-albumin antibody. The monovalent anti-properdin antibody including the sequence of SEQ ID NO: 59 may be linked by its N-terminus or C-terminus to the N-terminus or C-terminus of the monovalent anti-albumin antibody with a linker including the amino acid sequence of any one of SEQ ID NOs: 1-6, 63-64, and 100-101.

In some embodiments, a monovalent anti-properdin antibody including the amino acid sequence of SEQ ID NO: 60 is linked to a monovalent anti-albumin antibody. The monovalent anti-properdin antibody including the sequence of SEQ ID NO: 60 may be linked by its N-terminus or C-terminus to the N-terminus or C-terminus of the monovalent anti-albumin antibody with a linker including the amino acid sequence of any one of SEQ ID NOs: 1-6, 63-64, and 100-101.

In some embodiments, a monovalent anti-properdin antibody including the amino acid sequence of SEQ ID NO: 61 is linked to a monovalent anti-albumin antibody. The monovalent anti-properdin antibody including the sequence of SEQ ID NO: 61 may be linked by its N-terminus or C-terminus to the N-terminus or C-terminus of the monovalent anti-albumin antibody with a linker including the amino acid sequence of any one of SEQ ID NOs: 1-6, 63-64, and 100-101.

In some embodiments, a monovalent anti-properdin antibody including the amino acid sequence of SEQ ID NO: 60 is linked at its N-terminus to a monovalent anti-albumin antibody with a linker including the sequence of SEQ ID NO: 4.

In some embodiments, a monovalent anti-properdin antibody including the amino acid sequence of SEQ ID NO: 60 is linked at its N-terminus to a monovalent anti-albumin antibody with a linker including the sequence of SEQ ID NO: 5.

In some embodiments, a monovalent anti-properdin antibody including the amino acid sequence of SEQ ID NO: 60 is linked at its N-terminus to a monovalent anti-albumin antibody with a linker including the sequence of SEQ ID NO: 6.

In some embodiments, the bispecific construct includes the amino acid sequence of any one of SEQ ID NOs: 45-55, and 62.

In some embodiments, a monovalent anti-properdin antibody is linked at its N-terminus to a monovalent anti-albumin antibody with a linker including the sequence of SEQ ID NO: 1.

In some embodiments, a monovalent anti-properdin antibody is linked at its N-terminus to a monovalent anti-albumin antibody with a linker including the sequence of SEQ ID NO: 2.

In some embodiments, a monovalent anti-properdin antibody is linked at its N-terminus to a monovalent anti-albumin antibody with a linker including the sequence of SEQ ID NO: 3.

In some embodiments, a monovalent anti-properdin antibody is linked at its N-terminus to a monovalent anti-albumin antibody with a linker including the sequence of SEQ ID NO: 4.

In some embodiments, a monovalent anti-properdin antibody is linked at its N-terminus to a monovalent anti-albumin antibody with a linker including the sequence of SEQ ID NO: 5.

In some embodiments, a monovalent anti-properdin antibody is linked at its N-terminus to a monovalent anti-albumin antibody with a linker including the sequence of SEQ ID NO: 6.

In some embodiments, a monovalent anti-properdin antibody is linked at its N-terminus to a monovalent anti-albumin antibody with a linker including the sequence of SEQ ID NO: 63.

In some embodiments, a monovalent anti-properdin antibody is linked at its N-terminus to a monovalent anti-albumin antibody with a linker including the sequence of SEQ ID NO: 64.

In some embodiments, a monovalent anti-properdin antibody is linked at its C-terminus to a monovalent anti-albumin antibody with a linker including the sequence of SEQ ID NO: 1.

In some embodiments, a monovalent anti-properdin antibody is linked at its C-terminus to a monovalent anti-albumin antibody with a linker including the sequence of SEQ ID NO: 2.

In some embodiments, a monovalent anti-properdin antibody is linked at its C-terminus to a monovalent anti-albumin antibody with a linker including the sequence of SEQ ID NO: 3.

In some embodiments, a monovalent anti-properdin antibody is linked at its C-terminus to a monovalent anti-albumin antibody with a linker including the sequence of SEQ ID NO: 4.

In some embodiments, a monovalent anti-properdin antibody is linked at its C-terminus to a monovalent anti-albumin antibody with a linker including the sequence of SEQ ID NO: 5.

In some embodiments, a monovalent anti-properdin antibody is linked at its C-terminus to a monovalent anti-albumin antibody with a linker including the sequence of SEQ ID NO: 6.

In some embodiments, a monovalent anti-properdin antibody is linked at its C-terminus to a monovalent anti-albumin antibody with a linker including the sequence of SEQ ID NO: 63.

In some embodiments, a monovalent anti-properdin antibody is linked at its C-terminus to a monovalent anti-albumin antibody with a linker including the sequence of SEQ ID NO: 64.

### Generation of Single Domain Antibodies

In one embodiment, compositions and methods use a single domain antibody that is a heavy chain variable domain (V_{H}, e.g., V_{HH}) or a light chain domain (V_{L}). Thus, one means of generating monovalent single domain antibodies specific for properdin is to amplify and express the V_{H} and V_{L} regions of the heavy chain and light chain gene sequences isolated, for example, from a hybridoma (e.g., a mouse hybridoma) that expresses anti-properdin monoclonal antibody. The boundaries of V_{H} and V_{L} domains are set out, for example, by Kabat et al. (Sequences of Proteins of Immunological Interest (National Institutes of Health, Bethesda, MD, 1991). The information regarding the boundaries of the V_{H} and V_{L} domains of heavy and light chain genes is used to design PCR primers that amplify the V domain from a heavy or light chain coding sequence encoding an antibody known to bind properdin. The amplified V domains are inserted into a suitable expression vector, e.g., pHEN-1 (Hoogenboom, H. et al., Nucleic Acids Res., 19:4133-7, 1991) and expressed, for example, as a fusion of the V_{H} and V_{L} in a scFv or other suitable monovalent format. The resulting polypeptide can then be screened for high affinity monovalent binding to properdin. Screening for binding can be performed by methods known in the art. Single domain antibodies can be generated using methods known in the art (WO2005118642; Ward, E. et al., Nature, 341:544-6, 1989; Holt, L. et al., Trends Biotechnol., 21:484-90, 2003). Each light chain domain may be either of the kappa or lambda subgroup. Methods for isolating V_{H} and V_{L} domains have been described in the art (EP0368684).

In one embodiment, the single domain antibody is obtained from a human, humanized rodent, camelid or shark. Any such single domain antibody can be optionally humanized. Humanization of camelid single domain antibodies requires the introduction and mutagenesis of a limited number of amino acids in a single polypeptide chain. This is in contrast to humanization of scFv, Fab, (Fab′)2 and IgG, which requires the introduction of amino acid changes in two chains, the light and the heavy chain and the preservation of the assembly of both chains. In some embodiments, the single domain antibody includes V_{HH} domains. In some embodiments, the V_{HH} domains correspond to the V_{HH} domains of naturally occurring heavy chain antibodies directed against properdin. Such V_{HH} sequences can be generated, for example, by suitably immunizing a species of camelid with properdin (i.e., so as to raise an immune response and/or heavy chain antibodies directed against properdin), by obtaining a suitable biological sample from said camelid (such as a blood sample, serum sample or sample of B-cells), and by generating V_{HH} sequences directed against properdin, starting from said sample, using any suitable technique known in the art (e.g., the gene encoding the single domain antibody may be cloned by single cell PCR, or the B-cell(s) encoding the single domain antibody may be immortalized by EBV transformation, or by fusion to an immortal cell line).

Alternatively, such naturally occurring V_{HH} domains against properdin, can be obtained from naive libraries of camelid V_{HH} sequences, for example by screening such a library using properdin, or at least one part, fragment, antigenic determinant or epitope thereof using one or more screening techniques known in the art (WO 99/37681, WO 01/90190, WO 03/025020 and WO 03/035694). Alternatively, improved synthetic or semi-synthetic libraries derived from naive V_{HH} libraries may be used, such as V_{HH} libraries obtained from naive V_{HH} libraries by techniques such as random mutagenesis and/or CDR shuffling (WO 00/43507). In a certain embodiment, a V_{HH} library is constructed and expressed on phages after infection with helper phages. After several rounds of bio-panning, single domain antibodies against human properdin can be isolated and efficiently expressed.

A library of fusion proteins including V_{HH} Or V_{HH} fragments can be displayed on a phage, phagemid, ribosome or suitable microorganism (such as yeast), to facilitate screening. Suitable methods, techniques and host organisms for displaying and screening (a set, collection or library of) fusion proteins including V_{HH} Or V_{HH} fragments are known in the art (WO 03/054016; Hoogenboom, H., Nat. Biotechnol., 23:1105-16, 2005).

In an additional embodiment, the method for generating fusion proteins including V_{HH} Or V_{HH} fragment sequences includes at least the steps of: a) providing a collection or sample of cells derived from a species of camelid that express immunoglobulin sequences; b) screening the collection or sample of cells for (i) cells that express an immunoglobulin sequence that can bind to and/or have affinity for properdin; and (ii) cells that express heavy chain antibodies, in which substeps (i) and (ii) can be performed essentially as a single screening step or in any suitable order as two separate screening steps, to provide at least one cell that expresses a heavy chain antibody that can bind to and/or has affinity for properdin; and c) either (i) isolating from the cell the V_{HH} sequence present in the heavy chain antibody; or (ii) isolating from the cell a nucleic acid sequence that encodes the V_{HH} sequence present in the heavy chain antibody, followed by expressing the V_{HH} domain.

The method for generating an amino acid sequence directed against properdin can include at least the steps of: a) providing a set, collection or library of nucleic acid sequences encoding heavy chain antibodies or V_{HH} sequences; b) screening the set, collection or library of nucleic acid sequences for nucleic acid sequences that encode a heavy chain antibody or a fusion protein including the V_{HH} sequence that can bind to and/or has affinity for properdin; and c) isolating the nucleic acid sequence, followed by expressing the V_{HH} sequence present in the heavy chain antibody or by expressing the fusion protein including the V_{HH} sequence, respectively.

Other suitable methods and techniques for obtaining the single domain antibodies and/or nucleic acids encoding the same, starting from naturally occurring V_{H} sequences or V_{HH} sequences may, for example, include combining one or more parts of one or more naturally occurring V_{HH} sequences (such as one or more framework region (FR) sequences and/or CDR sequences), one or more parts of one or more naturally occurring V_{HH} sequences (such as one or more framework region sequences or CDR sequences), and/or one or more synthetic or semi-synthetic sequences, in a suitable manner, so as to provide a monovalent anti-properdin single domain antibody or a nucleotide sequence or nucleic acid encoding the same. Nucleotide sequences encoding framework sequences of V_{HH} or single domain antibodies are known in the art and may alternatively be obtained polymerase chain reaction (PCR) starting from the nucleotide sequences obtained using the methods described herein. Such compositions can be suitably combined with nucleotide sequences that encode the desired CDRs (for example, by PCR assembly using overlapping primers), to provide a single domain antibody, or antibody fragment fused with a regulator of the alternative complement pathway or fragment thereof.

### Generation of Antibody Fragments

Antibody fragments that recognize the same epitope as a parent antibody can be generated by known techniques. For example, antibody fragments can be prepared by proteolytic hydrolysis of an antibody or by expression in E. coli of the DNA coding for the fragment. The antibody fragments are antigen binding portions of an antibody, such as Fab, F(ab′)₂, and scFV can be obtained by pepsin or papain digestion of whole antibodies by conventional methods or by genetic engineering techniques.

An antibody fragment can be produced by enzymatic cleavage of antibodies with pepsin to provide a 100 kDa fragment denoted F(ab′)₂. This fragment can be further cleaved using a thiol reducing agent, and optionally a blocking group for the sulfhydryl groups resulting from cleavage of disulfide linkages, to produce 50 kDa Fab′ monovalent fragments. Alternatively, an enzymatic cleavage using papain produces two monovalent Fab fragments and an Fc fragment directly (U.S. Pat. Nos. 4,036,945 and 4,331,647; Nisonoff, A. et al., Arch. Biochem. Biophys., 89:230-44, 1960; Porter, R., Biochem. J., 73:119-26, 1959; Edelman et al., in Methods in Enzymology Vol. I, page 422 (Academic Press 1967), and Coligan et al., Current Protocols in Immunology, Vol. 1, pages 2.8.1-2.8.10 and 2.10.-2.10.4 (John Wiley & Sons 1991).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

Another form of an antibody fragment is a peptide coding for a single complementarity-determining region (CDR). CDR peptides can be obtained by constructing genes encoding the CDR of an antibody of interest. Such genes are prepared, for example, by using the polymerase chain reaction to synthesize the variable region from RNA of antibody producing cells (Larrick, J & Fry, K. METHODS—a companion to Methods in Enzymology Volume: New Techniques in Antibody Generation, 2:106-110, 1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles And Applications, Birch et al., (eds.), pages 137-185 (Wiley-Liss, Inc. 1995)).

Other antibody fragments, for example single domain antibody fragments, are known in the art and may be used in the claimed constructs (Muyldermans, S. et al., Trends Biochem. Sci., 26:230-5, 2001; Yau, K. et al., J. Immunol. Methods, 281:161-75, 2003; Maass, D. et al., J. Immunol. Methods, 324:13-25, 2007). The V_{HH} may have potent antigen binding capacity and can interact with novel epitopes that are inaccessible to conventional V_{H}-V_{L} pairs. Camelidae may be immunized with known antigens, such as properdin, and V_{HH}s can be isolated that bind to and neutralize the target antigen.

### Screening Monovalent Antibodies for Antigen Binding

Library screening methods can be used to identify monovalent properdin-specific binding antibodies or fragments. Phage display technology provides an approach for the selection of antibodies that bind a desired target (e.g., human properdin) from among large, diverse repertoires of antibodies (Smith, G., Science, 228:1315-7, 1985; Scott, J. & Smith, G., Science, 249:386-90, 1990; McCafferty, J. et al., Nature, 348:552-4, 1990). These phage-antibody libraries can be grouped into two categories: natural libraries that use rearranged V genes harvested from human B-cells (Marks, J. et al., J. Mol. Biol., 222:581-97, 1991; Vaughan, T. et al., Nat. Biotechnol., 14:309-14, 1996) or synthetic libraries whereby germline V gene segments or other antibody polypeptide coding sequences are ‘rearranged’ in vitro (Hoogenboom, H. & Winter, G., J. Mol. Biol., 227:381-8, 1992; Nissim, A. et al., EMBO J., 13:692-8, 1994; Griffiths, A. et al., EMBO J., 13:3245-60, 1994; de Kruif, J. et al., J. Mol. Biol., 248:97-105, 1995) or where synthetic CDRs are incorporated into a single rearranged V gene (Barbas, C. et al., Proc. Natl. Acad. Sci. USA, 89:4457-61, 1992). Methods involving genetic display packages (e.g., phage display, polysome display) are suited for the selection of monovalent properdin-specific antibody constructs because they generally express only monovalent fragments, rather than whole, divalent antibodies, on the display packages. Methods for the preparation of phage display libraries displaying various antibody fragments are described in the preceding references and, for example, in U.S. Pat. No. 6,696,245, which is incorporated herein by reference in its entirety.

Following expression of a repertoire of single domain antibodies on the surface of phage, selection is performed by contacting the phage repertoire with immobilized target antigen (e.g., properdin), washing to remove unbound phage, and propagation of the bound phage, the whole process frequently referred to as “panning.” This process is applicable to the screening of monovalent single domain antibodies and antibody fragments that can be expressed on a display library (e.g., scFv, Fab, (Fab′)2, and V_{HH}; Harrison, J. et al., Meth. Enzymol., 267:83-109, 1996). Alternatively, phages are pre-selected for the expression of properly folded member variants by panning against an immobilized generic ligand (e.g., protein A or protein L) that is only bound by folded members (WO 99/20749). This has the advantage of reducing the proportion of non-functional members, thereby increasing the proportion of members likely to bind a target antigen. The screening of phage antibody libraries is generally described, for example, by.

Screening is commonly performed using purified antigen immobilized on a solid support, for example, plastic tubes or wells, or on a chromatography matrix, for example Sepharose™ (Pharmacia). Screening or selection can also be performed on complex antigens, such as the surface of cells (Marks, J. et al., Biotechnology (NY), 11:1145-9, 1993; de Kruif, J. et al., Proc. Natl. Acad. Sci. USA, 92:3938-42, 1995). Another alternative involves selection by binding biotinylated antigen in solution, followed by capture on streptavidin-coated beads. V_{HH} coding sequences are known in the art and may be used to construct camelid V_{HH} phage display libraries, which can be used for antibody fragment isolation by bio-panning techniques known in the art.

### Expression of Anti-Properdin Antibodies

The manipulation of nucleic acids can be carried out in recombinant vectors. As used herein, “vector” refers to a discrete element that is used to introduce heterologous DNA into cells for the expression and/or replication thereof. Methods to select or construct and, subsequently, use such vectors are known to one of skill in the art. Numerous vectors are publicly available, including bacterial plasmids, bacteriophage, artificial chromosomes and episomal vectors. Such vectors may be used for simple cloning and mutagenesis. A vector is selected to accommodate a polypeptide coding sequence of a desired size. A suitable host cell is transformed with the vector after in vitro cloning manipulations. Each vector contains various functional components, which generally include a cloning (or “polylinker”) site and an origin of replication. An expression vector can further comprise one or more of the following: enhancer element, promoter, transcription termination and signal sequences—each positioned in the vicinity of the cloning site such that they are operatively linked to the gene encoding the polypeptide.

Both cloning and expression vectors generally contain nucleic acid sequences that enable the vector to replicate in one or more selected host cells. Typically cloning vectors comprise sequence elements that enable the vector to replicate independently of the host chromosomal DNA and includes origins of replication or autonomously replicating sequences. Such sequences are known for a variety of bacteria, yeast and viruses.

For screened libraries described herein, the vectors can be expression vectors that enable the expression of a polypeptide library member. Selection is performed by separate propagation and expression of a single clone expressing the polypeptide library member or by use of any selection display system. For bacteriophage display, phage or phagemid vectors can be used. Phagemid vectors have an E. coli origin of replication (for double stranded replication) and also a phage origin of replication (for production of single-stranded DNA).

### Purification and Concentration of Monovalent Antibodies

Monovalent antibodies secreted into the periplasmic space or into the medium of bacteria are harvested and purified according to known methods (Skerra, A. & Plückthun, A., Science, 240:1038-41, 1988; and Breitling, F. et al. (Gene, 104:147-53, 1991) describe the harvest of antibody polypeptides from the periplasm; Better, M. et al. (Science, 240:1041-3, 1988) describe harvest from the culture supernatant). For some antibody polypeptides, purification can also be achieved by binding to generic ligands, such as protein A or Protein L. Alternatively, the variable domains can be expressed with a peptide tag, e.g., the Myc, HA or 6×His tags, which facilitates purification by affinity chromatography. If necessary, monovalent anti-properdin antibodies are concentrated by any of several methods well known in the art, including, for example, ultrafiltration, diafiltration and tangential flow filtration. The process of ultrafiltration uses semi-permeable membranes and pressure to separate molecular species on the basis of size and shape. The pressure is provided by gas pressure or by centrifugation. By selection of a molecular weight cutoff smaller than the target antibody (usually ⅓ to ⅙ the molecular weight of the target polypeptide, the anti-properdin antibody is retained when solvent and smaller solutes pass through the membrane.

### Pharmaceutical Compositions, Dosage and Administration

The antibodies described herein can be incorporated into pharmaceutical compositions suitable for administration to a subject. Typically, the pharmaceutical composition includes a monovalent anti-properdin antibody, antibody derivative or fragment thereof and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The term “pharmaceutically acceptable carrier” excludes tissue culture medium including bovine or horse serum. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol or sodium chloride in the composition. Pharmaceutically acceptable substances include minor amounts of auxiliary substances such as wetting or emulsifying agents, preservatives or buffers, which enhance the shelf life or effectiveness of the antibody.

The compositions as described herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The final form depends on the intended mode of administration and therapeutic application. Typical compositions are in the form of injectable or infusible solutions, such as compositions similar to those used for passive immunization of humans with other antibodies. The composition(s) can delivered by, for example, parenteral injection (e.g., intravenous, subcutaneous, intraperitoneal, intramuscular).

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the monovalent anti-properdin antagonist in the required amount in an appropriate solvent with one or a combination of ingredients enumerated above, as required, followed by filter sterilization. Generally, dispersions are prepared by incorporating the monovalent anti-properdin antagonist into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The antibodies described herein can be administered by a variety of methods known in the art, although for many therapeutic applications, the preferred route/mode of administration is intravenous injection or infusion. The polypeptide can also be administered by intramuscular or subcutaneous injection.

As will be appreciated by the skilled artisan, the route and/or mode of administration will vary depending upon the desired results. In certain embodiments, the antibody may be prepared with a carrier that will protect the antibody against rapid release, such as a controlled release formulation, including implants, transdermal patches, and microencapsulated delivery systems. Monovalent single domain antibodies are suited for formulation as extended release preparations due, in part, to their small size—the number of moles per dose can be significantly higher than the dosage of, for example, full sized antibodies. Biodegradable, biocompatible polymers can be used, such as ethylene vinyl acetate, polyanhydrides, polyglycolic acid, collagen, polyorthoesters and polylactic acid. Prolonged absorption of injectable compositions can be attained by including in the composition an agent that delays absorption, for example, monostearate salts and gelatin. Many methods for the preparation of such formulations are known to those skilled in the art (e.g., Sustained and Controlled Release Drug Delivery Systems, J. R. Robinson, ed., Marcel Dekker, Inc., New York, 1978). Methods applicable to the controlled or extended release of antibodies such as the monovalent single domain antibodies disclosed herein are known (U.S. Pat. Nos. 6,306,406 and 6,346,274; U.S. Patent Application Nos: US20020182254 and US20020051808, the entire teachings of each of which are incorporated herein by reference).

In certain embodiments, a monovalent anti-properdin antibody, antibody derivative or fragment thereof can be orally administered, for example, with an inert diluent or an assimilable edible carrier. To administer a composition described herein by a method other than parenteral administration, it may be necessary to coat the compound with, or co-administer the compound with a material to prevent its inactivation.

Additional active compounds can also be incorporated into the compositions. In certain embodiments, a monovalent anti-properdin antibody, antibody derivative or fragment thereof is co-formulated with and/or co-administered with one or more additional therapeutic agents. For example, a monovalent anti-properdin antibody, antibody derivative or fragment thereof can be co-formulated and/or co-administered with one or more additional antibodies that bind other targets (e.g., antibodies that bind regulators of the alternative complement pathway). Such combination therapies may utilize lower dosages of the administered therapeutic agents, thus avoiding possible toxicities or complications associated with the various monotherapies. Additionally, the compositions described herein can be co-formulated or co-administered with other therapeutic agents to ameliorate side effects of administering the compositions described herein (e.g., therapeutic agents that minimize risk of infection in an immunocompromised environment, for example, anti-bacterial agents, anti-fungal agents and anti-viral agents).

The pharmaceutical compositions can include a “therapeutically effective amount” or a “prophylactically effective amount” of a monovalent anti-properdin antagonist (e.g., an antibody or derivative or fragment thereof). A “therapeutically effective amount” refers to an amount effective, at dosages and for periods of time necessary, to achieve the desired therapeutic result. A therapeutically effective amount of the antibody can vary according to factors such as the disease state, age, sex, and weight of the individual, and the ability of the monovalent anti-properdin antagonist to elicit a desired response in the individual. A “prophylactically effective amount” refers to an amount effective, at dosages and for periods of time necessary, to achieve the desired prophylactic result. In some embodiments, a prophylactic dose is used in subjects prior to or at an earlier stage of disease where the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. It is to be noted that dosage values can vary with the type and severity of the condition to be alleviated. It is to be further understood that for any particular subject, specific dosage regimens should be adjusted over time according to the individual need and the professional judgment of the administering clinician.

A non-limiting range for a therapeutically or prophylactically effective amount of a monovalent anti-properdin antibody, antibody derivative or fragment thereof is 0.1-20 mg/kg, more preferably 1-10 mg/kg. It is to be noted that dosage values can vary with the type and severity of the condition to be alleviated. It is to be further understood that for any particular subject, specific dosage regimens should be adjusted over time according to the individual need and the professional judgment of the administering clinician.

The efficacy of treatment with a monovalent anti-properdin antibody, antibody derivative or fragment thereof as described herein is judged by the skilled clinician on the basis of improvement in one or more symptoms or indicators of the disease state or disorder being treated. An improvement of at least 10% (increase or decrease, depending upon the indicator being measured) in one or more clinical indicators is considered “effective treatment,” although greater improvements are preferred, such as 20%, 30%, 40%, 50%, 75%, 90%, or even 100%, or, depending upon the indicator being measured, more than 100% (e.g., two-fold, three-fold, ten-fold, etc., up to and including attainment of a disease-free state).

### Use of Monovalent Anti-Properdin Antibodies

The compositions described herein can be used in methods of treating a disease or disorder mediated by alternative complement pathway dysfunction in an individual in need of such treatment, the method including administering to the individual a therapeutically effective amount of a composition that includes a monovalent anti-properdin antibody, antibody derivative or fragment thereof, preferably a composition including a single human immunoglobulin variable domain that binds human properdin. In one embodiment, the monovalent anti-properdin antibodies, antibody derivatives or fragments thereof described herein are useful in treating diseases mediated by alternative complement pathway dysregulation by inhibiting the alternative complement pathway activation in a mammal (e.g., a human). Such disorders include, without limitation, systemic lupus erythromatosus and lupus nephritis, rheumatoid arthritis, antiphospholipid (aPL) Ab syndrome, glomerulonephritis, paroxysmal nocturnal hemoglobinuria (PNH) syndrome, inflammation, organ transplantation, intestinal and renal I/R injury, asthma (e.g., severe asthma), atypical hemolytic-uremic syndrome (aHUS), spontaneous fetal loss, DDD, Macular degeneration, TTP, IgA nephropathy (Berger's disease), C3 glomerulopathy (C3G), Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMD), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, dialysis (cardiovascular risk), cardiovascular disease, placental malaria, APS pregnancy loss, membranoproliferative (MP) glomerulonephritis, membranous nephritism, encephalitis, brain injury, NMDA receptor antibody encephalitis, malaria hemolytic crisis, abdominal aortic aneurysm (AAA), and thoracoabdominal aortic aneurysm (TAA).

### EXAMPLES

The following examples are put forth so as to provide those of ordinary skill in the art with a disclosure and description of how the methods and compounds claimed herein are performed, made. They are intended to be purely exemplary and are not intended to limit the scope of the disclosure.

### Example 1. Generation of V_{HH}-His In-Fusion Cloning Vector

The pBNJ391 vector was digested with the restriction enzymes BstEII and EcoRI to remove the hinge and Fc. The vector was gel purified, which produced a 1000 bp release product. Annealed oligos UDEC6629/6630 were cloned into the pBNJ391 vector with BstEII/EcoRI. The annealed oligos contained the following sequences:

UDEC 6629 forward primer:
(SEQ ID NO: 65)
GTCACCGTGTCGAGCCATCATCACCATCATCACTGATGAG
UDEC 6630 reverse primer:
(SEQ ID NO: 66)
AATTCTCATCATTTGTCATCATCATCCTTATAGTCGCTCGACACG

The final vector contained a BstEII-6×His-EcoRI site.

Next, the pNGH0320 vector was digested with XhoI/BstEII (producing a 13 bp release product) and column purified. Next, a V_{HH} phage clone template was used to PCR amplify an insert. Forward primer UDEC 6438 (GTCCACTCCCTCGAGGTGCAGCTGGTGGAGTCTGGG; SEQ ID NO: 67) and reverse primer UDEC 6442 (GCTCGACACGGTGACCTGGGTCCCCTGGCCCCA; SEQ ID NO: 68), were used. The PCR products were purified and subsequently used in an In-Fusion protocol for cloning.

The pBNJ391 vector was digested with BstEII/EcoRI (50 ng/μL). Both complementary oligonucleotides were re-suspended at the same molar concentration, using TE Buffer. Equal volumes of both complementary oligonucleotides (at equimolar concentration) were mixed in a 1.5 ml tube. The tube was placed in a standard heat block at 90-95° C. for 3-5 minutes. The tube was removed from the apparatus and allowed to cool to room temperature (or at least below 30° C.). The tube was stored on ice or at 4° C. until further use.

A mix of 1 μL insert DNA (from the above nucleotide used for ligation to pBNJ391), 2 μL of pBNJ391 (EcoRI/BstEII, 100 ng), 1 μL of 10× Ligase Buffer (NEB B0202S Lot: 1091410), 1 μL of T4 DNA Ligase (NEB M0202L Lot: 0671502), and 5 μL of water formed the ligation reaction. The ligation reaction was incubated for 30 minutes at room temperature. 1 μL of the ligation reaction was transformed into 30 μL of DH10 chemical competent cells (InVitrogen 18297 Lot #1552241) and 750 μL of SOC (NEBB9020S Lot #2971403) was added. The tube was shaken for 1 hour at 37° C. and 10 μL and 100 μL were plated on an LB-carb/glucose plate. Plates were incubated over the weekend at room temperature.

Colonies were picked for PCR for insertion of the 6×His into pNGH0320. Eight colonies were screened and pBNJ391 was used as a negative control. 300 μL of TB/Carb/Glucose culture was added to isolated colonies and grown at 37° C. Forward primer UDEC5276 (CATAATAGCTGACAGACTAACAGACTG; SEQ ID NO: 69) and reverse primer UDEC1977 (CGAAACAAGCGCTCATGAGCCCGAAGT; SEQ ID NO: 70), were used. For a 20 μL PCR reaction, DNA from a single colony was added to 10 μL Go Taq Green PCR Mix, 0.2 μL Forward Primer (100 μM), 0.2 μL Reverse Primer (100 μM), and 9.6 μL H₂O, totaling 20 μL. PCR conditions are as follows: 95° C. for 3 minutes, 95° C. for 20 seconds, 50° C. for 20 seconds, and 72° C. for 1 minute 15 seconds. The cycle was repeated 30 times, followed by incubation at 72° C. for 5 minutes, and 4° C. until further use. 5 μL of the PCR product was mixed with 15 μL of water and run on a 2% E-gel. Two clones matched the predicted size. Plasmid maxi prep was performed using the overnight cultures with the Promega maxi prep kit.

To clone anti-properdin V_{HH} antibodies in V_{HH}-His tag format using in-fusion ligation of V_{HH} into pNGH0320, PCR was used to generate the V_{HH} insert with UDEC 6438-Infusion Forward Primer and UDEC 6442-Infusion reverse Primer for amplification of V_{H} phagemids from the Llama anti-properdin library pLNJ with an XhoI site for cloning into pNGH0317 by infusion. For a 60 μL PCR reaction, 30 μL 2× phusion PCR mix (NEB M0531s Lot: 0211412), 1 μL of bacterial culture, 0.1 μL Forward Primer UDEC 6438 (100 μM), 0.1 μL Reverse Primer UDEC 6442 (100 μM), and 28.8 μL H₂O, totaling 20 μL. PCR conditions are as follows: 98° C. for 3 minutes, 98 C for 10 seconds, 52 C for 15 seconds, 72 C for 30-60 seconds, followed by 72° C. for 5 minutes. The cycle was repeated 30 times and held at 4 C until further use. 5 μL of the PCR product was mixed with 15 μL of water and were run on a 2% E-gel. All clones matched the predicted size. Clones were pooled in reactions of eight and column purified using the Promega Wizard® SV Gel and PCR Clean-Up System, according to manufacturer's instructions. Plasmid maxi prep was performed using the overnight cultures with the Promega maxi prep kit.

For ligation of the insert, 2 μL of 5× In-Fusion HD Enzyme Premix (Clontech 639650 Lot: 1501713A), 2.5 μL of Vector pNGH0320 (XhoI/BstEII) 39.1 ng/μL (100 ng), 1 μL of purified PCR fragment (10-200 ng), and 4.5 μL of water formed the ligation reaction. The ligation reaction was incubated for 15 minutes at 50° C.

For transformation, Stellar™ competent cells (Clontech) were thawed in an ice bath just before use. After thawing, cells were mixed gently to ensure even distribution, and then 50 μL of competent cells were moved into a 14 mL round bottom tube (falcon tube). 1 μL (less than 5 ng of DNA) was added to the cells. The tube was placed on ice for 30 min. Next, the cells were heat shocked for exactly 45 sec at 42° C. Tubes were then placed on ice for 1-2 min. SOC medium was added to bring the final volume to 500 μL (SOC medium was warmed to 37 C before using). The tube was incubated while shaking (160-225 rpm) for 1 hr at 37° C. 10 μL of the solution was then place on LB plate containing carbenicillin. The plate was incubated overnight at 37 C.

A colony PCR screen was performed for insertion of the 24 V_{HH} colonies for each pool. A total of 48 clones were picked for each pool. Vector pNGH0320.1 was used as a positive control. Forward primer UDEC5276 and reverse primer UDEC1977 were used. For a 20 μL PCR reaction, DNA from a single colony was added to 10 μL Go Taq Green PCR Mix, 0.2 μL Forward Primer (100 μM), 0.2 μL Reverse Primer (100 μM), and 9.6 μL H₂O, totaling 20 μL. PCR conditions are as follows: 95° C. for 3 minutes, 95° C. for 20 seconds, 50° C. for 20 seconds, and 72° C. for 1 minute 15 seconds. Repeat for 30 cycles, followed by 72° C. for 5 minutes, and hold 4° C. until further use. 5 μL of the PCR products was mixed with 15 μL of water and were run on a 2% E-gel. Sequence analysis was performed on all 48 clones.

Preliminary screening of an immunization-biased llama V_{HH} phage display library resulted in identification of 192 V_{HH}s that were ELISA positive for binding properdin. 57 V_{HH}s were cloned and expressed with a 6× histidine tag. Of these, 34 V_{HH}s were Octet-positive for binding properdin. A summary is shown below in Table 1.

TABLE 1
Summary of screening assay

| | | | Cross-reactive | | Cross-reactive | |
|---|---|---|---|---|---|---|
| | | | screening | | screening | |
| | Screening | | on mouse | | on mouse | |
| | on h-fP | | and human fP | | and human fP | |
| | Standard | | Standard | | Standard | |
| | Procedure | NGS | Procedure | NGS | Procedure | NGS |
| ELISA positive | 193 | | 233 | | 284 | |
| Unique sequences | 72 | NA | 134 | NA | 90 | NA |
| (>3 different | | | | | | |
| a.a in CDR-3 | | | | | | |
| Cloned and | 57 | NA | NA | NA | NA | NA |
| expressed (with | | | | | | |
| 6xhistidine tag) | | | | | | |
| Octet positive | 34 | NA | NA | NA | NA | NA |
| Hemolysis positive | 4 | NA | NA | NA | NA | NA |
| (by using the | | | | | | |
| purified VHH) | | | | | | |

Four functional V_{HH}s were found to effectively inhibit alternative complement pathway-mediated hemolysis and are shown below in Table 2.

TABLE 2
Anti-properdin V_{HH} sequences

| Clone | |
|---|---|
| ID | V_{HH} sequence |
| AB005 | QVQVVESGGGLRQTGGSLRL |
| | SCTASGRIFEVNMMAVVYRQ |
| | APGKQRELVAEISRVGTTVY |
| | ADSVKGRFTISRDSAKNTVT |
| | LQMNSLKSEDTAVYYCNALQ |
| | YDRYGGAEYWGQGTQVTVSS |
| | (SEQ ID NO: 58) |
| AB006 | QVQLAESGGGLVQAGDSLKL |
| | SCTASGRIFEVNMMAVVYRQ |
| | APGKDRELVAEISRVGTTTY |
| | ADSVKGRFTISRDSAKNTVT |
| | LQMNSLKSEDTAVYYCNALQ |
| | YSRYGGAEYWGQGTQVTVSG |
| | (SEQ ID NO: 59) |
| AB007 | QVQLVESGGGLVQAGGSLRL |
| | SCAASGRISSIIHMAVVYRQ |
| | APGKQRELVAEISRVGTTVY |
| | ADSVKGRFTISRDDAKNTVT |
| | LQMNSLKPEDTAVYYCNALQ |
| | YEKHGGADYWGQGTQVTVSG |
| | (SEQ ID NO: 60) |
| AB008 | QVQLVESGGGLRQTGESLRL |
| | SCTASGRIFEVNMMAVVYRQ |
| | APGKQRELVAEISRVGTTTY |
| | ADSVKGRFTISRDSAKNTVT |
| | LQMNSLKSEDTAVYYCNALQ |
| | YDRYGGAEYWGQGTQVTVSG |
| | (SEQ ID NO: 61) |

### Example 2. Binding of Anti-Properdin V_{HH} Antibodies to Human Properdin

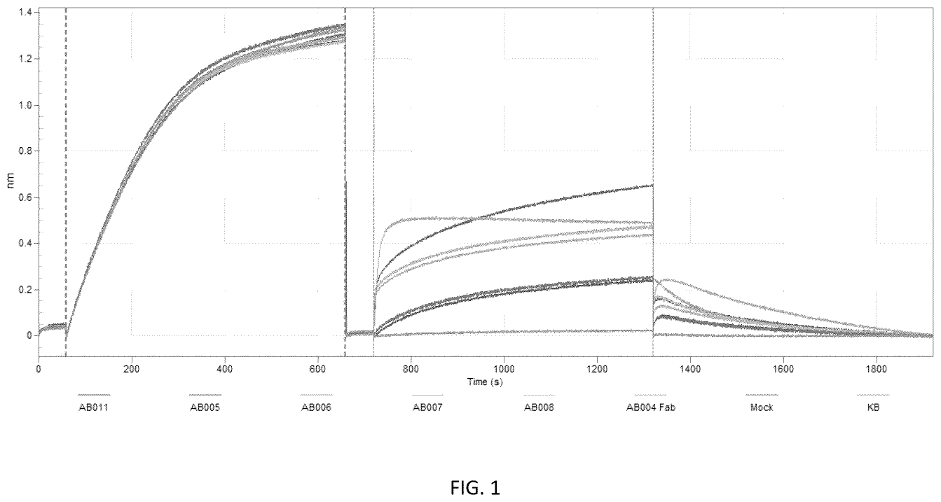

FIG. 1 shows kinetic binding measurements can be performed on an Octet instrument (FortéBio Inc.). All washes, dilutions, and measurements are performed in Kinetic buffer (FortéBio cat 185032) with the plate shaking at 1000 rpm. Streptavidin Biosensors (Forte Bio Cat: 18-5019 lot: 1405301) were equilibrated in Kinetic buffer for 10 min and then loaded with 50 nm of biotinylated human properdin. For the association phase, 10 μg/mL of selected anti-properdin antibody or kinetics buffer blank was added to the biosensors preloaded with biotinylated human properdin respectively. Results show binding of AB005, AB006, AB007 and AB008 to human properdin.

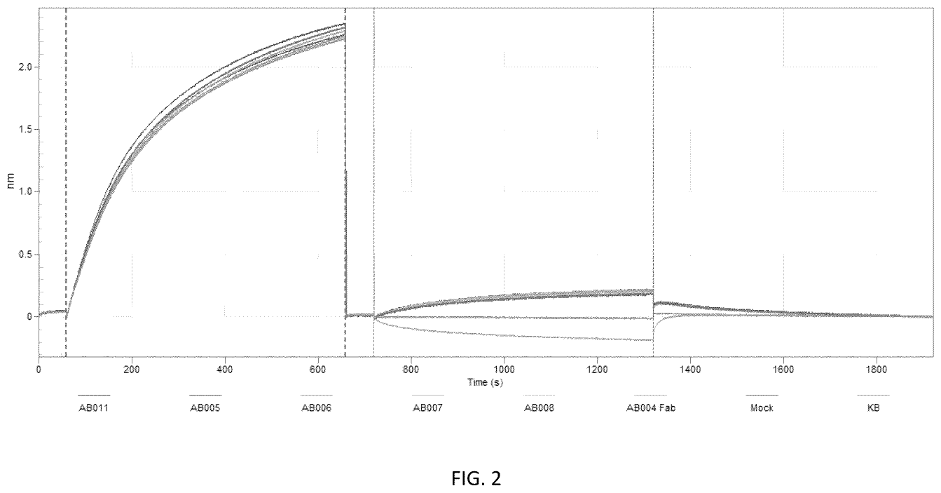

FIG. 2 shows kinetic binding measurements can be performed on an Octet instrument (FortéBio Inc). All washes, dilutions, and measurements are performed in Kinetic buffer (FortéBio cat 185032) with the plate shaking at 1000 rpm. Streptavidin Biosensors (Forte Bio Cat: 18-5019 lot: 1405301) were equilibrated in Kinetic buffer for 10 min and then loaded with 50 nm of biotinylated mouse properdin. For the association phase, 10 μg/mL of selected anti-properdin antibody or kinetics buffer blank was added to the biosensors preloaded with biotinylated human properdin respectively. Results show weak or no binding of AB005, AB006, AB007, and AB008.

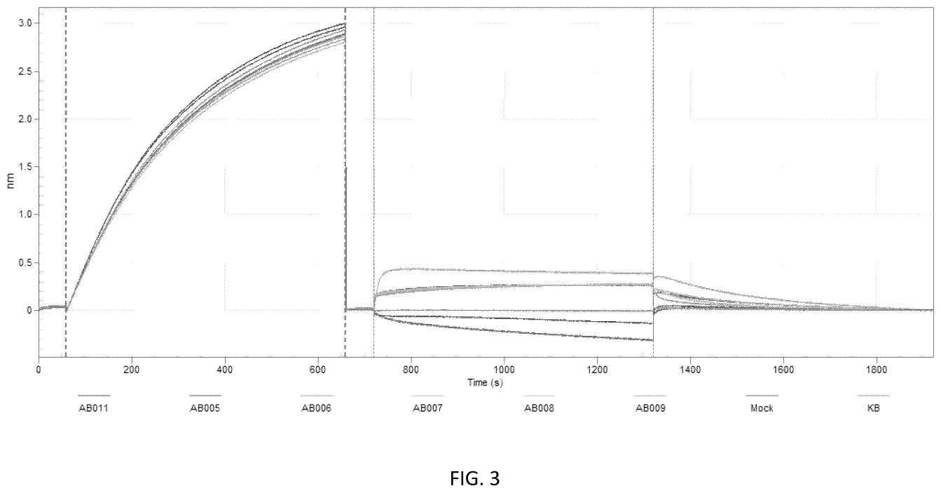

FIG. 3 shows kinetic binding measurements can be performed on an Octet instrument (FortéBio Inc.). All washes, dilutions, and measurements are performed in Kinetic buffer (FortéBio cat 185032) with the plate shaking at 1000 rpm. Streptavidin Biosensors (Forte Bio Cat: 18-5019 lot: 1405301) were equilibrated in Kinetic buffer for 10 min and then loaded with 50 nm of biotinylated cynomolgus properdin. For the association phase, 10 μg/mL of selected anti-properdin antibody or kinetics buffer blank was added to the biosensors preloaded with biotinylated human properdin respectively. Results show weak binding of AB005, AB006, AB007, and AB008 to cynomolgus properdin.

### Example 3. Alternative Complement Hemolysis Assay

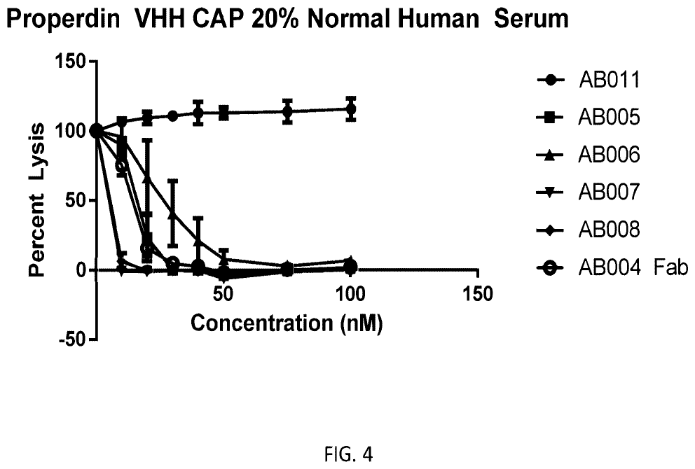

FIG. 4 shows an alternative complement pathway-mediated hemolysis assay based on the formation of a terminal complement-complex on the surface of the rabbit red blood cell (rRBC). As a result of the formation of this complex, the rRBCs are lysed. Agents that inhibit the formation of complement complexes are expected to inhibit cellular lysis. Various anti-properdin antigen binding fragments were tested to evaluate the effect on cellular lysis mediated by alternative complement activation. An “assay plate” was prepared by diluting 40% normal human serum with Gelatin veronal buffer (GVB) supplemented with 10 mM EGTA and 10 mM MgCl₂ (e.g., 1600 μL normal human serum into 2400 μLGVB supplemented with 10 mM EGTA and 10 mM MgCl₂). 50 μL of this solution was distributed into each well of the assay plate (polystyrene). Next, the dilution plate (polypropylene) was prepared by adding 50 μL/well of 2×mAbs (e.g., anti-properdin Fab) in GVB supplemented with 10 mM EGTA and 10 mM MgCl₂ at a concentration ranging from 0-100 nM to appropriate wells. As a positive control rabbit red blood cells were incubated in distilled water (100% lysis of cells) and for the negative control the red blood cells were incubated in GVB with 10 mM EDTA and 10 mM MgCl₂, respectively (0% lysis of cells).

50 μL/well was transferred from the dilution plate to assay plate. The assay plate was left at room temperature while proceeding to the next step. 400 μL of rRBCs were washed 4 times, each with 1 mL of GVB supplemented with 10 mM EGTA and 10 mM MgCl₂. rRBCs were spun at 2600 rpm for 1 minute after each wash. After the final wash, rRBCs were resuspend to a volume of 400 μL by adding 300 μL GVB supplemented with 10 mM EGTA and 10 mM MgCl₂. 50 μL of washed rRBCs were resuspended to 1 mL with GVB supplemented with 10 mM EGTA and 10 mM MgCl₂. 30 μL of this dilute solution was added to 100 μL of the prepared sample in the assay plate, yielding 1.5×10⁶ cells/well. The plate was incubated for 30 minutes at 37° C. The plate was then centrifuged at 1000×g for 5 min and 85 μL of the supernatant was transferred to a flat bottom 96 well plate. Hemolysis was determined by measuring OD at 415 nm. A progressive decrease in light scatter (due to the lysis of intact cells) was measured at 415 nm as a function of concentration. For the calculation, the total inhibition was calculated at each concentration of the anti-properdin V_{HH} and the results were expressed as a percentage of unlisted controls.

### Example 4. Binding Kinetics of Monovalent Anti-Properdin V_{HH} Antibodies to Properdin

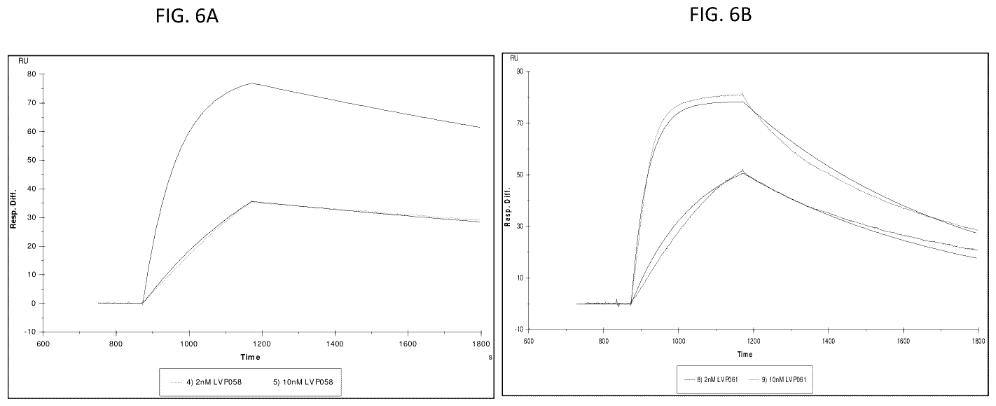

In FIG. 6, anti-properdin V_{HH} antibodies AB007 and AB008, respectively, were run at known concentrations over an immobilized sensor surface. Response level (RU) was plotted against time in the sensorgrams.

The binding affinities of anti-properdin V_{HH} antibodies were determined. The results are summarized in Table 3 below.

TABLE 3
Binding kinetics

| | | kₐ | k_{d} | K_{D} | | |
|---|---|---|---|---|---|---|
| Sample | | (1/Ms) | (1/s) | (M) | Chi² | |
| AB007 | | 1.04e6 | 3.59e−4 | 3.44e−10 | 0.36 | |
| AB008 | | 2.11e6 | 1.69e−3 | 8.03e−10 | 5.29 | |
| | | kₐ | k_{d} | K_{D} | | Com- |
| Sample | Antigen | (1/Ms) | (1/s) | (M) | Chi² | ments |
| AB009 | Human | 1.69E+06 | 4.33E−05 | 2.55E−11 | 0.18 | Good |
| | Properdin | | | | | Fit |
| AB010 | Human | 1.09E+07 | 7.17E−05 | 6.59E−12 | 0.11 | Good |
| | Properdin | | | | | Fit |

### Example 5. Binding Kinetics of Bispecific Anti-Properdin Antibodies to Properdin and Alternative Complement Hemolysis Assay

Bispecific constructs were created based on the anti-properdin constructs described above linked with a linker to an anti-albumin construct. The binding to properdin and alternative complement hemolysis was measured in similar assays as described above. The sequences of the constructs are shown in Table 4 below.

TABLE 4
anti-properdin construct sequences

| Molecule | Description | AA Sequence |
|---|---|---|
| TPP- | anti-Albumin | EVQLVESGGGLVKPGGSLRL |
| 2225 | LVP058 | SCAASGRPVSNYAAAWFRQA |
| | (G4S)₃ Linker | PGKEREFVSAINWQKTATYA |
| | | DSVKGRFTISRDNAKNSLYL |
| | | QMNSLRAEDTAVYYCAAVFR |
| | | VVAPKTQYDYDYWGQGTLVT |
| | | VSSGGGGSGGGGSGGGGSLE |
| | | VQLVESGGGLVQAGGSLRLS |
| | | CAASGRISSIIHMAWYRQAP |
| | | GKQRELVAEISRVGTTVYAD |
| | | SVKGRFTISRDDAKNTVTLQ |
| | | MNSLKPEDTAVYYCNALQYE |
| | | KHGGADYWGQGTQVTVSS |
| | | (SEQ ID NO: 45) |
| TPP- | Humanized anti- | EVQLVESGGGLVKPGGSLRL |
| 2951 | Albumin LVP058 | SCAASGRPVSNYAAAWFRQA |
| | (G4S)₃ Linker | PGKEREFVSAINWQKTATYA |
| | (7-backmutations) | DSVKGRFTISRDNAKNSLYL |
| | | QMNSLRAEDTAVYYCAAVFR |
| | | VVAPKTQYDYDYWGQGTLVT |
| | | VSSGGGGSGGGGSGGGGSEV |
| | | QLLESGGGLVQPGGSLRLSC |
| | | AASGRISSIIHMAWFRQAPG |
| | | KERELVSEISRVGTTVYADS |
| | | VKGRFTISRDNSKNTLYLQM |
| | | NSLKPEDTAVYYCNALQYEK |
| | | HGGADYWGQGTLVTVSS |
| | | (SEQ ID NO: 46) |
| TPP- | anti- Albumin | EVQLVESGGGLVKPGGSLRL |
| 3071 | LVP058 | SCAASGRPVSNYAAAWFRQA |
| | (G4D)₂ (G4) | PGKEREFVSAINWQKTATYA |
| | Linker | DSVKGRFTISRDNAKNSLYL |
| | | QMNSLRAEDTAVYYCAAVFR |
| | | VVAPKTQYDYDYWGQGTLVT |
| | | VSSGGGGDGGGGDGGGGEVQ |
| | | LVESGGGLVQAGGSLRLSCA |
| | | ASGRISSIIHMAWYRQAPGK |
| | | QRELVAEISRVGTTVYADSV |
| | | KGRFTISRDDAKNTVTLQMN |
| | | SLKPEDTAVYYCNALQYEKH |
| | | GGADYWGQGTQVTVSS |
| | | (SEQ ID NO: 47) |
| TPP- | anti- Albumin | EVQLVESGGGLVKPGGSLRL |
| 3072 | LVP058 | SCAASGRPVSNYAAAWFRQA |
| | (G4E)₂ | PGKEREFVSAINWQKTATYA |
| | (G4) Linker | DSVKGRFTISRDNAKNSLYL |
| | | QMNSLRAEDTAVYYCAAVFR |
| | | VVAPKTQYDYDYWGQGTLVT |
| | | VSSGGGGEGGGGEGGGGEVQ |
| | | LVESGGGLVQAGGSLRLSCA |
| | | ASGRISSIIHMAWYRQAPGK |
| | | QRELVAEISRVGTTVYADSV |
| | | KGRFTISRDDAKNTVTLQMN |
| | | SLKPEDTAVYYCNALQYEKH |
| | | GGADYWGQGTQVTVSS |
| | | (SEQ ID NO: 48) |
| TPP- | Humanized anti- | EVQLVESGGGLVKPGGSLRL |
| 3261 | Albumin LVP058 | SCAASGRPVSNYAAAWFRQA |
| | (G4S)₃ Linker | PGKEREFVSAINWQKTATYA |
| | (3-backmutations) | DSVKGRFTISRDNAKNSLYL |
| | | QMNSLRAEDTAVYYCAAVFR |
| | | VVAPKTQYDYDYWGQGTLVT |
| | | VSSGGGGSGGGGSGGGGSEV |
| | | QLVESGGGLVQPGGSLRLSC |
| | | AASGRISSIIHMAWVRQAPG |
| | | KQRELVSEISRVGTTVYADS |
| | | VKGRFTISRDNSKNTLYLQM |
| | | NSLRAEDTAVYYCNALQYEK |
| | | HGGADYWGQGTLVTVSS |
| | | (SEQ ID NO: 49) |
| TPP- | Humanized anti- | EVQLVESGGGLVKPGGSLRL |
| 3341 | Albumin LVP058 | SCAASGRPVSNYAAAWFRQA |
| | (G4D)₂ | PGKEREFVSAINWQKTATYA |
| | (G4) Linker | DSVKGRFTISRDNAKNSLYL |
| | (7-backmutations) | QMNSLRAEDTAVYYCAAVFR |
| | | WAPKTQYDYDYWGQGTLVTV |
| | | SSGGGGDGGGGDGGGGEVQL |
| | | LESGGGLVQPGGSLRLSCAA |
| | | SGRISSIIHMAWFRQAPGKE |
| | | RELVSEISRVGTTVYADSVK |
| | | GRFTISRDNSKNTLYLQMNS |
| | | LKPEDTAVYYCNALQYEKHG |
| | | GADYWGQGTLVTVSS |
| | | (SEQ ID NO: 50) |
| TPP- | Humanized anti- | EVQLVESGGGLVKPGGSLRL |
| 3342 | Albumin LVP058 | SCAASGRPVSNYAAAWFRQA |
| | (G4E)₂ | PGKEREFVSAINWQKTATYA |
| | (G4) Linker | DSVKGRFTISRDNAKNSLYL |
| | (7-backmutations) | QMNSLRAEDTAVYYCAAVFR |
| | | VVAPKTQYDYDYWGQGTLVT |
| | | VSSGGGGEGGGGEGGGGEVQ |
| | | LLESGGGLVQPGGSLRLSCA |
| | | ASGRISSIIHMAWFRQAPGK |
| | | ERELVSEISRVGTTVYADSV |
| | | KGRFTISRDNSKNTLYLQMN |
| | | SLKPEDTAVYYCNALQYEKH |
| | | GGADYWGQGTLVTVSS |
| | | (SEQ ID NO: 51) |
| TPP- | Humanized anti- | EVQLVESGGGLVKPGGSLRL |
| 3343 | Albumin LVP058 | SCAASGRPVSNYAAAWFRQA |
| | (G4D)₂ | PGKEREFVSAINWQKTATYA |
| | (G4) Linker | DSVKGRFTISRDNAKNSLYL |
| | (3-backmutations) | QMNSLRAEDTAVYYCAAVFR |
| | | VVAPKTQYDYDYWGQGTLVT |
| | | VSSGGGGDGGGGDGGGGEVQ |
| | | LVESGGGLVQPGGSLRLSCA |
| | | ASGRISSIIHMAWVRQAPGK |
| | | QRELVSEISRVGTTVYADSV |
| | | KGRFTISRDNSKNTLYLQMN |
| | | SLRAEDTAVYYCNALQYEKH |
| | | GGADYWGQGTLVTVSS |
| | | (SEQ ID NO: 52) |
| TPP- | Humanized anti- | EVQLVESGGGLVKPGGSLRL |
| 3344 | Albumin-LVP058 | SCAASGRPVSNYAAAWFRQA |
| | (G4E)₂ | PGKEREFVSAINWQKTATYA |
| | (G4) Linker | DSVKGRFTISRDNAKNSLYL |
| | (3-backmutations) | QMNSLRAEDTAVYYCAAVFR |
| | | VVAPKTQYDYDYWGQGTLVT |
| | | VSSGGGGEGGGGEGGGGEVQ |
| | | LVESGGGLVQPGGSLRLSCA |
| | | ASGRISSIIHMAWVRQAPGK |
| | | QRELVSEISRVGTTVYADSV |
| | | KGRFTISRDNSKNTLYLQMN |
| | | SLRAEDTAVYYCNALQYEKH |
| | | GGADYWGQGTLVTVSS |
| | | (SEQ ID NO: 62) |
| TP- | LVP058_hG2-G4- | LEVQLVESGGGLVQAGGSLR |
| 2221 | V_{HH} on silent | LSCAASGRISSIIHMAWYRQ |
| | human Fc | APGKQRELVAEISRVGTTVY |
| | | ADSVKGRFTISRDDAKNTVT |
| | | LQMNSLKPEDTAVYYCNALQ |
| | | YEKHGGADYWGQGTQVTVSS |
| | | RKCCVECPPCPAPPVAGPSV |
| | | FLFPPKPKDTLMISRTPEVT |
| | | CVVVDVSQEDPEVQFNWYVD |
| | | GVEVHNAKTKPREEQFNSTY |
| | | RVVSVLTVLHQDWLNGKEYK |
| | | CKVSNKGLPSSIEKTISKAK |
| | | GQPREPQVYTLPPSQEEMTK |
| | | NQVSLTCLVKGFYPSDIAVE |
| | | WESNGQPENNYKTTPPVLDS |
| | | DGSFFLYSRLTVDKSRWQEG |
| | | NVFSCSVMHEALHNHYTQKS |
| | | LSLSLGK |
| | | (SEQ ID NO: 53) |
| TP- | LVP058_ | LEVQLVESGGGLVQAGGSLR |
| 2222 | hG1_noC1q-V_{HH} | LSCAASGRISSIIHMAWYRQ |
| | on human Fc | APGKQRELVAEISRVGTTVY |
| | without C1q | ADSVKGRFTISRDDAKNTVT |
| | binding | LQMNSLKPEDTAVYYCNALQ |
| | | YEKHGGADYWGQGTQVTVSS |
| | | PKSCDKTHTCPPCPAPELLG |
| | | GPSVFLFPPKPKDTLMISRT |
| | | PEVTCVVVDVSHEDPEVKFN |
| | | WYVDGVEVHNAKTKPREEQY |
| | | NSTYRVVSVLTVLHQDWLNG |
| | | KEYKCAVSNKALPAPIEKTI |
| | | SKAKGQPREPQVYTLPPSRE |
| | | EMTKNQVSLTCLVKGFYPSD |
| | | IAVEWESNGQPENNYKTTPP |
| | | VLDSDGSFFLYSKLTVDKSR |
| | | WQQGNVFSCSVMHEALHNHY |
| | | TQKSLSLSPGK |
| | | (SEQ ID NO: 54) |
| TPP- | LVP058 (G4S)₃- | LEVQLVESGGGLVQAGGSLR |
| 2224 | anti-A1b- | LSCAASGRISSIIHMAWYRQ |
| | tandem | APGKQRELVAEISRVGTTVY |
| | V_{HH} | ADSVKGRFTISRDDAKNTVT |
| | | LQMNSLKPEDTAVYYCNALQ |
| | | YEKHGGADYWGQGTQVTVSS |
| | | GGGGSGGGGSGGGGSEVQLV |
| | | ESGGGLVKPGGSLRLSCAAS |
| | | GRPVSNYAAAWFRQAPGKER |
| | | EFVSAINWQKTATYADSVKG |
| | | RFTISRDNAKNSLYLQMNSL |
| | | RAEDTAVYYCAAVFRVVAPK |
| | | TQYDYDYWGQGTLVTVSS |
| | | (SEQ ID NO: 55) |
| TPP- | Anti-properdin | Light Chain Sequence: |
| 2223 | control antibody | DIQMTQSPSSLSASVGDRVT |
| | without C1q | ITCRASQDISFFLNWYQQKP |
| | binding domain | GKAPKLLIYYTSRYHSGVPS |
| | | RFSGSGSGTDFTLTISSLQP |
| | | EDFATYYCQHGNTLPWTFGQ |
| | | GTKVEIKRTVAAPSVFIFPP |
| | | SDEQLKSGTASWCLLNNFYP |
| | | REAKVQWKVDNALQSGNSQE |
| | | SVTEQDSKDSTYSLSSTLTL |
| | | SKADYEKHKVYACEVTHQGL |
| | | SSPVTKSFNRGEC |
| | | (SEQ ID NO: 56) |
| | | Heavy Chain Sequence: |
| | | QVQLVQSGAEVKKPGASVKV |
| | | SCKASGYIFTNYPIHWVRQA |
| | | PGQGLEWMGFIDPGGGYDEP |
| | | DERFRDRVTMTRDTSTSTVY |
| | | MELSSLRSEDTAVYYCARRG |
| | | GGYYLDYWGQGTLVTVSSAS |
| | | TKGPSVFPLAPSSKSTSGGT |
| | | AALGCLVKDYFPEPVTVSWN |
| | | SGALTSGVHTFPAVLQSSGL |
| | | YSLSSWTVPSSSLGTQTYIC |
| | | NVNHKPSNTKVDKRVEPKSC |
| | | DKTHTCPPCPAPELLGGPSV |
| | | FLFPPKPKDTLMISRTPEVT |
| | | CVVVDVSHEDPEVKFNWYVD |
| | | GVEVHNAKTKPREEQYNSTY |
| | | RVVSVLTVLHQDWLNGKEYK |
| | | CAVSNKALPAPIEKTISKAK |
| | | GQPREPQVYTLPPSREEMTK |
| | | NQVSLTCLVKGFYPSDIAVE |
| | | WESNGQPENNYKTTPPVLDS |
| | | DGSFFLYSKLTVDKSRWQQG |
| | | NVFSCSVMHEALHNHYTQKS |
| | | LSLSPGK |
| | | (SEQ ID NO: 57) |

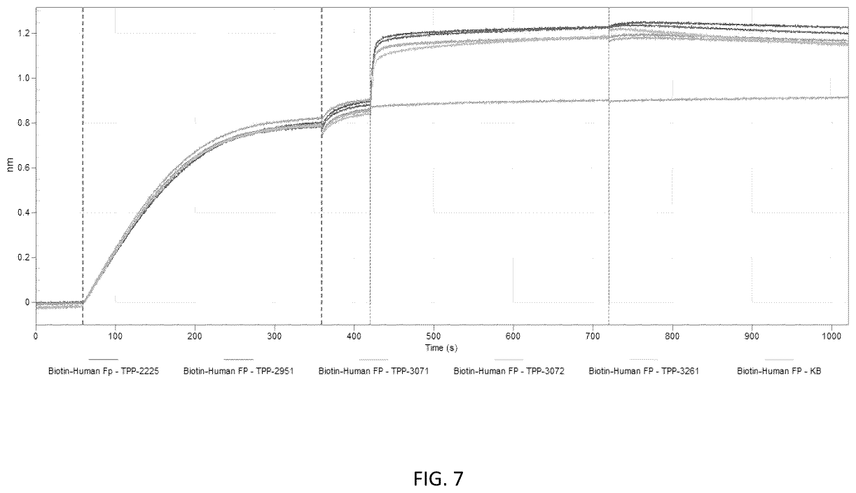

FIG. 7 shows kinetic binding measurements performed on an Octet instrument (FortéBio Inc.). All washes, dilutions, and measurements were performed in Kinetic buffer (FortéBio cat 185032) with the plate shaking at 1000 rpm. Streptavidin Biosensors (Forte Bio Cat: 18-5019 lot: 1405301) were equilibrated in Kinetic buffer for 10 min and then loaded with 50 nm of biotinylated human properdin. For the association phase, 10 μg/mL of selected anti-properdin antibody or kinetics buffer blank was added to the biosensors preloaded with biotinylated human properdin, respectively. Results show binding of TPP-2225, TPP-2591, TPP-3071, TPP-3072, TPP-3261 to human properdin. The results show strong binding by all constructs to human properdin.

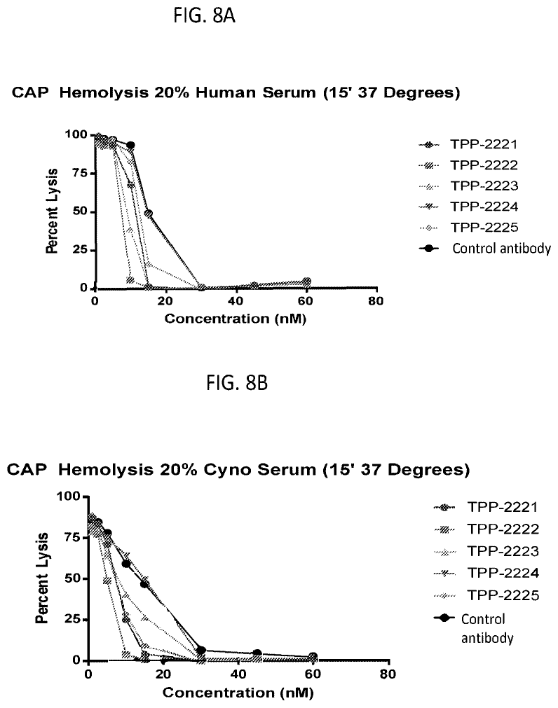

FIG. 8A-FIG. 8B show results of alternative complement pathway-mediated hemolysis assays based on the formation of a terminal complement-complex on the surface of the rabbit red blood cell (rRBC). As a result of the formation of this complex, the rRBCs are lysed. Agents that inhibit the formation of complement complexes are expected to inhibit cellular lysis. Various bispecific anti-properdin antigen binding constructs were tested to evaluate the effect on cellular lysis mediated by alternative complement activation. An “assay plate” was prepared by diluting 40% normal human serum with Gelatin veronal buffer (GVB) supplemented with 10 mM EGTA and 10 mM MgCl₂ (e.g., 1600 μL normal human serum into 2400 μLGVB supplemented with 10 mM EGTA and 10 mM MgCl₂). 50 μL of this solution was distributed into each well of the assay plate (polystyrene). Next, the dilution plate (polypropylene) was prepared by adding 50 μL/well of 2×mAbs (e.g., anti-properdin Fab) in GVB supplemented with 10 mM EGTA and 10 mM MgCl₂ at a concentration ranging from 0-100 nM to appropriate wells. As a positive control rabbit red blood cells were incubated in distilled water (100% lysis of cells) and for the negative control the red blood cells were incubated in GVB with 10 mM EDTA and 10 mM MgCl₂, respectively (0% lysis of cells).

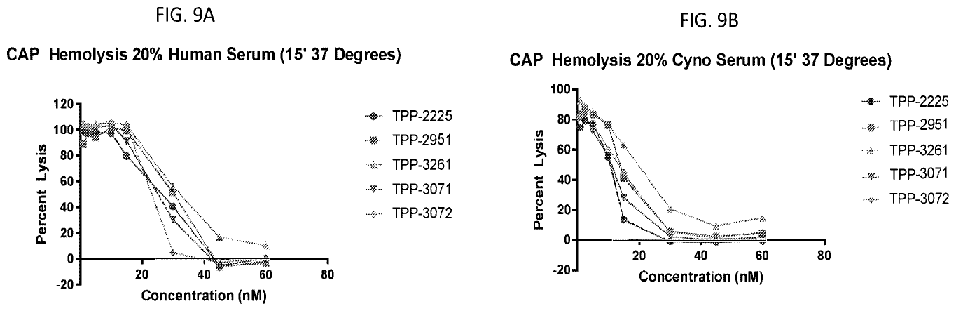

50 μL/well was transferred from the dilution plate to assay plate. The assay plate was left at room temperature while proceeding to the next step. 400 μL of rRBCs were washed 4 times, each with 1 mL of GVB supplemented with 10 mM EGTA and 10 mM MgCl₂. rRBCs were spun at 2600 rpm for 1 minute after each wash. After the final wash, rRBCs were resuspend to a volume of 400 μL by adding 300 μL GVB supplemented with 10 mM EGTA and 10 mM MgCl₂. 50 μL of washed rRBCs were resuspended to 1 mL with GVB supplemented with 10 mM EGTA and 10 mM MgCl₂. 30 μL of this dilute solution was added to 100 μL of the prepared sample in the assay plate, yielding 1.5×10⁶ cells/well. The plate was incubated for 30 minutes at 37° C. The plate was then centrifuged at 1000×g for 5 min and 85 μL of the supernatant was transferred to a flat bottom 96 well plate. Hemolysis was determined by measuring OD at 415 nm. A progressive decrease in light scatter (due to the lysis of intact cells) was measured at 415 nm as a function of concentration. For the calculation, the total inhibition was calculated at each concentration of the anti-properdin antibody construct and the results were expressed as a percentage of unlisted controls. FIG. 8A-FIG. 8B show hemolysis mediated by TPP-2221, TP-2222, TP-2223, TP-2224, and TP-2225 in human (FIG. 8A) and cynomolgus (FIG. 8B) serum. The control antibody is an anti-properdin antibody. FIG. 9A-FIG. 9B show hemolysis mediated by TPP-2225, TPP-2951, TPP-3261, TPP-3071, and TPP-3072 in human (FIG. 9A) and cynomolgus (FIG. 9B) serum.

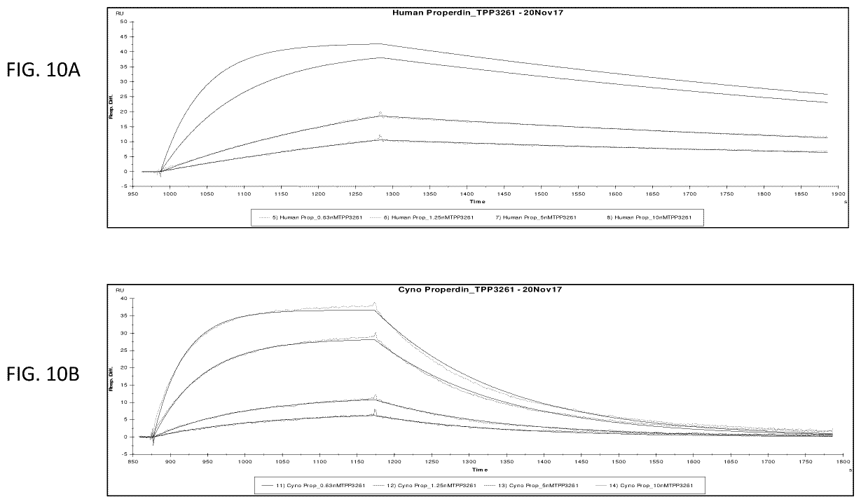

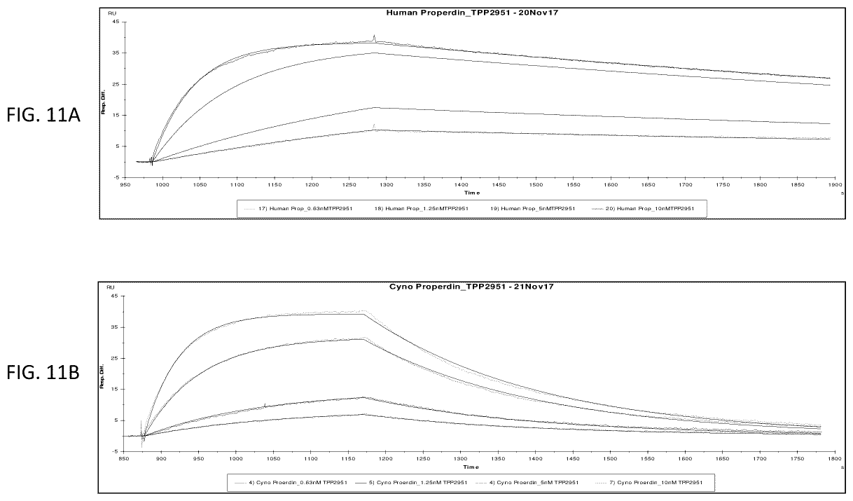

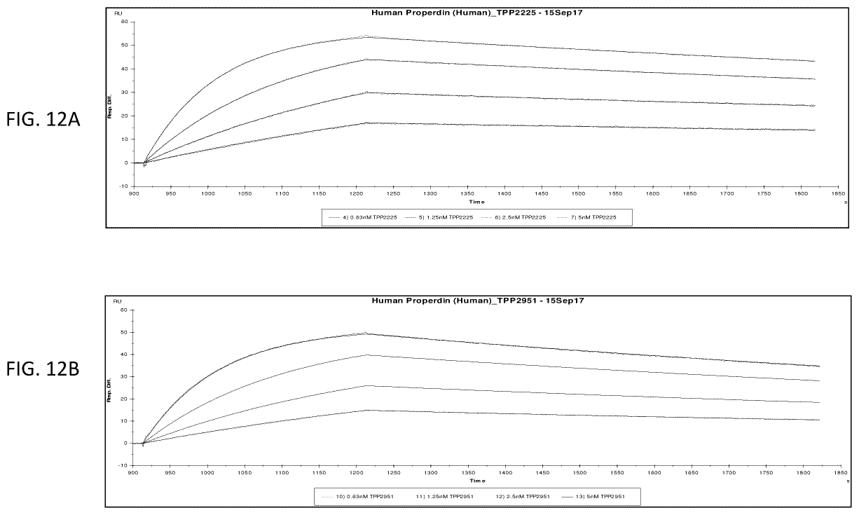

FIG. 10A-FIG. 10B, FIG. 11A-FIG. 11B, and FIG. 12A-FIG. 12B show the binding kinetics of TPP-3261, TPP-2951, and TPP-2225 to human and cynomolgus properdin.

The binding affinities and IC50 values are shown for each construct in the following Tables 5-9.

TABLE 5
Binding kinetics of bispecific constructs

| Molecule | IC50 (nM) | Description |
|---|---|---|
| Anti-properdin | 14.6-15.4 | Anti-properdin |
| control | | |
| TPP-2221 | 7.1-8.4 | LVP058_hG2-G4 |
| TPP-2222 | 5.1-5.8 | LVP058_hG1_noC1q |
| TPP-2223 | 8.4-13.4 | Anti-properdin |
| | | hG1_noC1q |
| TPP-2224 | 13.9-15.8 | LVP058-anti-Alb |
| TPP-2225 | 11.6-12.9 | anti-Alb-LVP058 |

TABLE 6
Binding kinetics of bispecific constructs

| | Affinity | | | |
|---|---|---|---|---|
| | pH 7.4 | Human | Cynomolgus | |
| Molecule | (nM) | IC50 (nM) | IC50 (nM) | Description |
| TPP-2225 | 1.72E−10 | 20.04 to | 11.19 to 12.3 | Non-humanized |
| | | 64.49 | | (G4S)3 Linker |
| TPP-2951 | 3.01E−10 | — | 13.76 to 15.89 | Humanized (7 back |
| | | | | mutations) |
| TPP-3261 | 4.85E−10 | 28.82 to | 14.8 to 23.83 | Humanized (3 back |
| | | 30.96 | | mutations) |
| TPP-3071 | — | 22.28 to | 10.66 to 14.58 | Non-humanized |
| | | 29.36 | | (G4D)2 G4 Linker |
| TPP-3072 | — | — | 13.06 to 18.65 | Non-humanized |
| | | | | (G4E)2 G4 Linker |

TABLE 7
Binding kinetics of bispecific constructs

| | | | Cynomolgus | | | | Cynomolgus | |
|---|---|---|---|---|---|---|---|---|
| | Human Properdin | | Properdin | | Human Albumin | | Albumin | |
| Molecule | pH 7.4 | pH 6.0 | pH 7.4 | pH 6.0 | pH 7.4 | pH 6.0 | pH 7.4 | pH 6.0 |
| TPP-2225 | 1.72E−10 | 2.571E−9 | 1.979E−9 | No fit | 7.74e−10 | 6.46e− | 7.07e−9 | 2.30e−9 |
| | | | | | | 10 | | |
| TPP-2951 | 3.01E−10 | | 2.33E−9 | | | | | |
| TPP-3261 | 4.85E−10 | | 3.08E−9 | | | | | |

TABLE 8
Binding kinetics of bispecific constructs.

| | Properdin | kₐ | k_{d} | K_{D} | | Com- |
|---|---|---|---|---|---|---|
| Molecule | Type | (1/Ms) | (1/s) | (M) | Chi² | ments |
| TPP3261 | Human | 1.72e6 | 8.34e−4 | 4.85e−10 | 0.07 | Good Fit |
| TPP3261 | Cynomolgus | 1.91e6 | 5.87e−3 | 3.08e−9 | 0.24 | Good Fit |
| TPP2951 | Human | 1.78e6 | 5.74e−4 | 3.22e−10 | 0.06 | Good Fit |
| TPP2951 | Cynomolgus | 1.82e6 | 4.26e−3 | 2.33e−9 | 0.17 | Good Fit |

TABLE 9
Binding kinetics of bispecific constructs.

| | Properdin | kₐ | k_{d} | K_{D} | | |
|---|---|---|---|---|---|---|
| Molecule | Type | (1/Ms) | (1/s) | (M) | Chi² | Comments |
| TPP2225 | Human | 2.03e6 | 3.49e−4 | 1.72e−10 | 0.04 | Good Fit |
| TPP2951 | Human | 1.88e6 | 5.67e−4 | 3.01e−10 | 0.05 | Good Fit |

### OTHER EMBODIMENTS

All publications, patents, and patent applications mentioned in this specification are herein incorporated by reference to the same extent as if each independent publication or patent application was specifically and individually indicated to be incorporated by reference.

It will be understood that the compositions and methods described herein are capable of further modification(s), and this description is intended to include any variations, uses or adaptations following, in general, the principles disclosed herein-including such departures from the present disclosure that come within known or customary practice within the art that may be applied to the essential features.

1. An isolated monovalent antibody or antibody fragment thereof, wherein the antibody or antibody fragment thereof binds human properdin.

2. The antibody or fragment thereof of claim 1, wherein the antibody or fragment is a camelid antibody.

3. The antibody or fragment thereof of claim 1, wherein the antibody or fragment is a single-domain antibody.

4. The antibody or fragment thereof of claim 1, wherein the antibody or fragment binds to TSR0 and/or TSR1 of human properdin.

5. The antibody of fragment thereof of claim 1, wherein the antibody or fragment binds an epitope within the amino acid sequence LCQPCRSPRWSLWSTWAPCSVTCSEGSQLRYRRCVGWNGQ (SEQ ID NO: 8).

6. The antibody or fragment thereof of claim 1, wherein the antibody or fragment binds to mouse properdin with an affinity of less than 50 nM.

7. The antibody or fragment thereof of claim 1, wherein the antibody or fragment is humanized.

8. The antibody or fragment thereof of claim 1, wherein the antibody or fragment is linked to a second monovalent antibody or antibody fragment.

9. The antibody or fragment thereof of claim 8, wherein the second monovalent antibody or antibody fragment is humanized.

10. The antibody or fragment thereof of claim 8, wherein the antibody or fragment is linked to the second antibody or fragment by a linker.

11. The antibody of fragment thereof of claim 10, wherein the linker is a poly-glycine linker.

12. The antibody of fragment thereof of claim 11, wherein the poly-glycine linker comprises a GGGGD (SEQ ID NO: 63), a GGGGS (SEQ ID NO: 1), GGGGA (SEQ ID NO: 100), or a GGGGE (SEQ ID NO: 64) sequence.

13. The antibody or fragment thereof of claim 8, wherein the second monovalent antibody or antibody fragment specifically binds albumin.

14. The antibody or fragment thereof of claim 8, wherein the second monovalent antibody or fragment thereof is linked to the N-terminus of the antibody or antibody fragment that binds human properdin.

15. The antibody or fragment thereof of claim 8, wherein the antibody is linked to an albumin binding peptide.

16. The antibody or fragment thereof of claim 1, wherein the antibody or fragment comprises 3 CDRs with the following sequences:
a) a CDR-H1 comprising the amino acid sequence GRISSIIHMA (SEQ ID NO: 16);
b) a CDR-H2 comprising the amino acid sequence RVGTTVYADSVKG (SEQ ID NO: 12); and
c) a CDR-H3 comprising the amino acid sequence LQYEKHGGADY (SEQ ID NO: 17).

17. The antibody or fragment thereof of claim 1, wherein the antibody or fragment comprises at least one or all three CDRs selected from:
a) a CDR-H1 comprising the amino acid sequence GRIFEVNMMA (SEQ ID NO: 9);
b) a CDR-H2 comprising the amino acid sequence RVGTTX₁YADSVKG (SEQ ID NO: 10), wherein X₁ is a polar or a nonpolar amino acid; and
c) a CDR-H3 comprising the amino acid sequence LQYX₂RYGGAEY (SEQ ID NO: 11), wherein X₂ is a polar amino acid.

18. The antibody of claim 17, wherein CDR-H2 comprises the amino acid sequence RVGTTVYADSVKG (SEQ ID NO: 12).

19. The antibody of claim 18, wherein CDR-H3 comprises the amino acid sequence LQYDRYGGAEY (SEQ ID NO: 13).

20. The antibody of claim 17, wherein CDR-H2 comprises the amino acid sequence RVGTTTYADSVKG (SEQ ID NO: 15).

21. The antibody of claim 20, wherein CDR-H3 has the amino acid sequence LQYSRYGGAEY (SEQ ID NO: 14).

22. The antibody of claim 20, wherein CDR-H3 has the amino acid sequence LQYDRYGGAEY (SEQ ID NO: 13).

23. The antibody of claim 17, wherein CDR-H3 has the amino acid sequence LQYDRYGGAEY (SEQ ID NO: 13).

24. The antibody of claim 17, wherein CDR-H3 has the amino acid sequence LQYSRYGGAEY (SEQ ID NO: 14).

25. The antibody or fragment thereof of claim 1, wherein the antibody comprises 6 CDRs with the following sequences:
a) a CDR-H1 comprising the amino acid sequence GYIFTNYPIH (SEQ ID NO: 18);
b) a CDR-H2 comprising the amino acid sequence FIDPGGGYDEPDERFRD (SEQ ID NO: 19);
c) a CDR-H3 comprising the amino acid sequence RGGGYYLDY (SEQ ID NO: 20);
d) a CDR-L1 comprising the amino acid sequence RASQDISFFLN (SEQ ID NO: 21);
e) a CDR-L2 comprising the amino acid sequence YTSRYHS (SEQ ID NO: 22); and
f) a CDR-L3 comprising the amino acid sequence QHGNTLPWT (SEQ ID NO: 23).

26. The antibody or fragment thereof of claim 1, wherein the antibody comprises 6 CDRs with the following sequences:
a) a CDR-H1 comprising the amino acid sequence GFSLTTYGVH (SEQ ID NO: 24);
b) a CDR-H2 comprising the amino acid sequence VIWSGGDTDYNASFIS (SEQ ID NO: 25);
c) a CDR-H3 comprising the amino acid sequence NKDYYTNYDFTMDY (SEQ ID NO: 26);
d) a CDR-L1 comprising the amino acid sequence KSSQSVLYSSNQKNFLA (SEQ ID NO: 27);
e) a CDR-L2 comprising the amino acid sequence WASTRES (SEQ ID NO: 28); and
f) a CDR-L3 comprising the amino acid sequence HQYLSSYT (SEQ ID NO: 29).

27. The antibody or fragment thereof of claim 1, wherein the antibody comprises 6 CDRs with the following sequences:
a) a CDR-H1 comprising the amino acid sequence GYTFIDYWIE (SEQ ID NO: 30);
b) a CDR-H2 comprising the amino acid sequence EIFPGSGTINHNEKFKD (SEQ ID NO: 31);
c) a CDR-H3 comprising the amino acid sequence EGLDY (SEQ ID NO: 32);
d) a CDR-L1 comprising the amino acid sequence SASSSVSYIY (SEQ ID NO: 33);
e) a CDR-L2 comprising the amino acid sequence DTSTLAS (SEQ ID NO: 34); and
f) a CDR-L3 comprising the amino acid sequence QQWSRNPFT (SEQ ID NO:35).

28. The antibody or fragment thereof of claim 1, wherein the antibody comprises 6 CDRs with the following sequences:
a) a CDR-H1 comprising the amino acid sequence GFSLTSYGVH (SEQ ID NO: 36);
b) a CDR-H2 comprising the amino acid sequence VIWSGGSTDYNAAFIS (SEQ ID NO: 37);
c) a CDR-H3 comprising the amino acid sequence NKDFYSNYDYTMDY (SEQ ID NO: 38);
d) a CDR-L1 comprising the amino acid sequence KSSQSVLYSSNQKNFLA (SEQ ID NO: 27);
e) a CDR-L2 comprising the amino acid sequence WASTRES (SEQ ID NO: 28); and
f) a CDR-L3 comprising the amino acid sequence HQYLSSYT (SEQ ID NO: 29).

29. The antibody or fragment thereof of claim 1, wherein the antibody comprises 6 CDRs with the following sequences:
a) a CDR-H1 comprising the amino acid sequence GYTXTAYGIN (SEQ ID NO: 39);
b) a CDR-H2 comprising the amino acid sequence YIYIGNGYTDYNEKFKG (SEQ ID NO: 40);
c) a CDR-H3 comprising the amino acid sequence SGWDEDYAMDF (SEQ ID NO: 41);
d) a CDR-L1 comprising the amino acid sequence RASENIYSYLA (SEQ ID NO: 42);
e) a CDR-L2 comprising the amino acid sequence HAKTLAE (SEQ ID NO: 43); and
f) a CDR-L3 comprising the amino acid sequence QHHYGPPPT (SEQ ID NO: 44).

30. The antibody or fragment thereof of claim 1 or 8, wherein the antibody or fragment inhibits an activity of human properdin.

31. A method of treating a disease mediated by alternative complement pathway dysregulation, comprising administering an effective amount of the antibody of claim 1 or 8 to a patient in need thereof.

32. The method of claim 31, wherein the disease is autoimmune thrombotic thrombocytopenic purpura (TTP), hemolytic uremic syndrome (HUS), atypical hemolytic uremic syndrome (aHUS), or paroxysmal nocturnal hemoglobinuria (PNH).

33. A method of inhibiting alternative complement pathway membrane attack complex assembly, comprising administering an effective amount of the antibody of claim 1 or 8 to a patient in need thereof.

34. The method of claim 33, wherein the method inhibits alternative complement pathway dependent hemolysis.

35. A pharmaceutical composition comprising antibody or fragment thereof of claim 1 or 8, as an active in gradient, and a pharmaceutically acceptable carrier.

36. The antibody or fragment thereof of claim 1 or 8 for use as a medicament.

37. The antibody of fragment thereof for use according to claim 1 or 8 for use in treating a disease mediated by alternative complement pathway dysregulation.

38. The antibody of fragment thereof for use according to claim 37, wherein the disease is autoimmune thrombotic thrombocytopenia purpura (TTP), hemolytic uremic syndrome (HUS), atypical hemolytic uremic syndrome (aHUS), paroxysmal nocturnal hemoglobinuria (PNH), IgA nephropathy (Berger's disease), C3 glomerulopathy (C3G), asthma, Gaucher disease, Hidradentitis suppurativa, Behcet's disease, dermatomyositis, severe burn, early sepsis, pneumococcal meningitis, Alzheimer's disease, cancer metastasis, acute respiratory distress syndrome (ARDS), acute lung injury (ACI), transfusion-related lung injury (TRALI), hemodialysis induced thrombosis, epidermolysis bullosa acquisita (EBA), uveitis, Parkinson's disease, primary biliary atresia, antineutrophil cytoplasmic antibodies (ANCA) vasculitis, retinal degeneration, broad thrombotic microangiopathy (TMA), broad TMA (APS), hematopoietic stem cell therapy (HSCT) TMA, age-related macular degeneration (AMD), pre-eclampsia, hemolysis, elevated liver enzymes, and low platelet (HELLP) syndrome, multiple sclerosis, antiphospholipid syndrome (APS), relapsing polychondritis, ischemic injury, stroke, graft versus host disease (GvHD), chronic obstructive pulmonary disease (COPD), emphysema, atherosclerosis, acute coronary syndrome, hemorrhagic shock, rheumatoid arthritis, dialysis (cardiovascular risk), cardiovascular disease, placental malaria, antiphospholipid syndrome (APS) pregnancy loss, membranoproliferative (MP) glomerulonephritis, membranous nephritism, encephalitis, brain injury, N-methyl-D-aspartate (NMDA) receptor antibody encephalitis, malaria hemolytic crisis, abdominal aortic aneurysm (AAA), or thoracoabdominal aortic aneurysm (TAA).

39. An isolated antibody or fragment thereof, wherein the antibody comprises the following sequence:
(SEQ ID NO: 53)
LEVQLVESGGGLVQAGGSLRLSCAASGRISSIIHMAWYRQAPGKQRELV
AEISRVGTTVYADSVKGRFTISRDDAKNTVTLQMNSLKPEDTAVYYCNA
LQYEKHGGADYWGQGTQVTVSSRKCCVECPPCPAPPVAGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK.

40. An isolated antibody or fragment thereof, wherein the antibody comprises the following sequence:
(SEQ ID NO: 54)
LEVQLVESGGGLVQAGGSLRLSCAASGRISSIIHMAWYRQAPGKQRELV
AEISRVGTTVYADSVKGRFTISRDDAKNTVTLQMNSLKPEDTAVYYCNA
LQYEKHGGADYWGQGTQVTVSSPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCAVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPGK.
